SEQUENCE LISTING (30 sequences)

SEQ ID NO: 1 - protein, 10 aa, Homo sapiens
HKTDSFVGLM

SEQ ID NO: 2 - protein, 7 aa, Artificial sequence
  NKA-related sequences
  PEPTIDE (1)..(7): NKA
DSFVGLM

SEQ ID NO: 3 - protein, 8 aa, Artificial sequence
  NKA-related sequences
  PEPTIDE (1)..(8): NKA, X= NH - (R) - gamma - Lactam-Leu
KDSFVGXM

SEQ ID NO: 4 - protein, 7 aa, Artificial sequence
  NKA-related sequences
  PEPTIDE (1)..(7): NKA, X= beta- Ala
DSFVXLM

SEQ ID NO: 5 - protein, 7 aa, Artificial sequence
  NKA-related sequences
  PEPTIDE (1)..(7): NKA, X=Nle
DSFVGLX

SEQ ID NO: 6 - protein, 7 aa, Artificial sequence
  NKA-related sequences
  PEPTIDE (1)..(7): NKA, X= beta - Ala
DSFWXLM

SEQ ID NO: 7 - protein, 11 aa, Artificial sequence
  NKA-related sequences
  PEPTIDE (1)..(11): NKA
YHKTDSFVGLM

SEQ ID NO: 8 - protein, 8 aa, Artificial sequence
  NKA-related sequences
  PEPTIDE (1)..(8): NKA, X(position 4)= NH - (R) - Phe and X(position 7)= gamma - Lactam-Leu
KDSXVGXM

SEQ ID NO: 9 - protein, 7 aa, Artificial sequence
  NKA-related sequences
  PEPTIDE (1)..(7): NKA, X(position 6)=Methyl-Leu; X(position 7)=Nle
DKFVGXX

SEQ ID NO: 10 - protein, 398 aa, Homo sapiens
MGTCDIVTEANISSGPESNTTGITAFSMPSWQLALWATAYLALVLVAVTGNAIVIWIILAHRRMRTVTNYFIVNLALADLCMAAFNAAFNFVYASHNIWYFGRAFCYFQNLFPITAMFVSIYSMTAIAADRYMAIVHPFQPRLSAPSTKAVIAGIWLVALALASPQCFYSTVTMDQGATKCVVAWPEDSGGKTLLLYHLVVIALIYFLPLAVMFVAYSVIGLTLWRRAVPGHQAHGANLRHLQAMKKFVKTMVLVVLTFAICWLPYHLYFILGSFQEDIYCHKFIQQVYLALFWLAMSSTMYNPIIYCCLNHRFRSGFRLAFRCCPWVTPTKEDKLELTPTTSLSTRVNRCHTKETLFMAGDTAPSEATSGEAGRPQDGSGLWFGYGLLAPTKTHVEI

SEQ ID NO: 11 - protein, 384 aa, Mus musculus
MGAHASVTDTNILSGLESNATGVTAFSMPGWQLALWATAYLALVLVAVTGNATVIWIILAHERMRTVTNYFIINLALADLCMAAFNATFNFIYASHNIWYFGSTFCYFQNLFPVTAMFVSIYSMTAIAADRYMAIVHPFQPRLSAPSTKAVIAVIWLVALALASPQCFYSTITVDQGATKCVVAWPNDNGGKMLLLYHLVVFVLIYFLPLVVMFAAYSVIGLTLWKRAVPRHQAHGANLRHLQAKKKFVKAMVLVVVTFAICWLPYHLYFILGTFQEDIYYRKFIQQVYLALFWLAMSSTMYNPIIYCCLNHRFRSGFRLAFRCCPWGTPTEEDRLELTHTPSISRRVNRCHTKETLFMTGDMTHSEATNGQVGGPQDGEPAGP

SEQ ID NO: 12 - protein, 129 aa, Homo sapiens
MKILVALAVFFLVSTQLFAEEIGANDDLNYWSDWYDSDQIKEELPEPFEHLLQRIARRPKPQQFFGLMGKRDADSSIEKQVALLKALYGHGQISHKRHKTDSFVGLMGKRALNSVAYERSAMQNYERRR

SEQ ID NO: 13 - protein, 114 aa, Homo sapiens
MKILVALAVFFLVSTQLFAEEIGANDDLNYWSDWYDSDQIKEELPEPFEHLLQRIARRPKPQQFFGLMGKRDAGHGQISHKRHKTDSFVGLMGKRALNSVAYERSAMQNYERRR

SEQ ID NO: 14 - protein, 115 aa, Mus musculus
MKILVAVAVFFLVSTQLFAEEIDANDDLNYWSDWSDSDQIKEAMPEPFEHLLQRIARRPKPQQFFGLMGKRDAGHGQISHKRHKTDSFVGLMGKRALNSVAYERSAMQNYERRRK

SEQ ID NO: 15 - protein, 130 aa, Mus musculus
MKILVAVAVFFLVSTQLFAEEIDANDDLNYWSDWSDSDQIKEAMPEPFEHLLQRIARRPKPQQFFGLMGKRDADSSVEKQVALLKALYGHGQISHKRHKTDSFVGLMGKRALNSVAYERSAMQNYERRRK

SEQ ID NO: 16 - protein, 7 aa, Artificial sequence
  NKA-related sequences
  PEPTIDE (1)..(7): X(position 1)=S,K; X(position 2)=V,W; X(position 3)=G, beta - Ala; X(position 4)=Me-Leu, L, gamma - Lactam-Leu; X(position 5)=Nle, M
DXFXXXX

SEQ ID NO: 17 - DNA, 21 bp, Artificial sequence
  NKA-related sequences
  misc_feature (1)..(21): 36B4 Fwd
tcatccagcaggtgtttgaca

SEQ ID NO: 18 - DNA, 18 bp, Artificial sequence
  NKA-related sequences
  misc_feature (1)..(18): 36B4 Rev
ggcaccgaggcaacagtt

SEQ ID NO: 19 - DNA, 20 bp, Artificial sequence
  NKA-related sequences
  misc_feature (1)..(20): Tacr2 Fwd
gcctccccacaatgtttcta

SEQ ID NO: 20 - DNA, 20 bp, Artificial sequence
  NKA-related sequences
  misc_feature (1)..(20): Tacr2 Rev
tgaggacaaacaccaccaga

SEQ ID NO: 21 - protein, 10 aa, Artificial sequence
  NKA-related sequences
  PEPTIDE (1)..(10): NKA
SKTDSFVGLM

SEQ ID NO: 22 - protein, 10 aa, Artificial sequence
  NKA-related sequences
  PEPTIDE (1)..(10): X=norleucine
HKTDSFVGLX

SEQ ID NO: 23 - protein, 10 aa, Artificial sequence
  NKA-related sequences
  PEPTIDE (1)..(10): NKA
HKTESFVGLM

SEQ ID NO: 24 - protein, 10 aa, Artificial sequence
  NKA-related sequences
  PEPTIDE (1)..(10): X=norleucine
HKTESFVGLX

SEQ ID NO: 25 - protein, 9 aa, Artificial sequence
  NKA-related sequences
  PEPTIDE (1)..(9): NKA
KTDSFVGLM

SEQ ID NO: 26 - protein, 8 aa, Artificial sequence
  NKA-related sequences
  PEPTIDE (1)..(8): NKA
KDSFVGLM

SEQ ID NO: 27 - protein, 8 aa, Artificial sequence
  NKA-related sequences
  PEPTIDE (1)..(8): NKA
KESFVGLM

SEQ ID NO: 28 - protein, 11 aa, Artificial Sequence
  Peptide
  PEPTIDE (1)..(11): SP
RPKPQQFFGLM

SEQ ID NO: 29 - protein, 10 aa, Artificial sequence
  NKA-related sequences
  PEPTIDE (1)..(10): NKB
DMHDFFVGLM

SEQ ID NO: 30 - protein, 7 aa, Artificial sequence
  NKA-related sequences
  PEPTIDE (1)..(7): X(position 1)= E or D; X(position 2)= S or K; X(position 3)= V or W;X(position 4)= G or beta-alanine; X(position 5)= Methyl-Leucine or L or gamma - lactam-Leucine; X(position 6)= Nle or M
XXFXXXX

(19) United States
(12) Patent Application Publication
(10) Pub. No.: US 2024/0124524 A1
(43) Pub. Date: Apr. 18, 2024

(54) Agonist of Tacr2

(71) Applicant: University of Copenhagen, Copenhagen K (DK)

(72) Inventors: Zachary Gerhart-Hines, Valby (DK); Jakob Bondo Hansen, Copenhagen V (DK); Thue W. Schwartz, Frederiksberg (DK)

(21) Appl. No.: 17/768,525

(22) PCT Filed: Oct. 17, 2019
(86) PCT No.: PCT/EP2019/078201

§ 371 Date: Apr. 13, 2022

(51) Int. Cl. C07K 7/22; A61K 47/54; A61P 3/04; A61P 3/10; A61P 5/50

(52) U.S. Cl. CPC C07K 7/22; A61K 47/542; A61K 47/549; A61P 3/04; A61P 3/10; A61P 5/50

(57) ABSTRACT

The present disclosure relates to agonists of Tacr2, such as peptides agonist of Tacr2 and methods of using the same for treatment of insulin resistance, obesity and/or diabetes. The disclosure also relates to use of said agonists of Tacr2 for enhancement of energy consumption in an individual.

### FIELD OF INVENTION

The present disclosure relates to agonists of Tacr2, such as peptides agonist of Tacr2 and methods of using the same for treatment of obesity and/or diabetes. The disclosure also relates to use of said agonists of Tacr2 for enhancement of glucose and energy consumption in an individual.

### BACKGROUND OF INVENTION

There are approximately 2 billion overweight or obese and 350 million diabetic individuals in the world (according to the World Health Organization) and, despite continuous research, there is still a need for efficacious treatments.

One of the studied therapeutic strategies is activation of brown and beige/brite adipose tissue. Classical activation of brown and beige/brite adipose tissue by norepinephrine (NE) increases energy consumption in these cells through the beta-adrenergic receptors (β3ARs) (Cannon and Nedergaard, 2004). One of the main effectors of this brown and beige/brite adipose energy expenditure is uncoupling protein 1 (Ucp1), which dissipates the proton gradient to produce heat instead of ATP generation. In addition to activation of brown and beige/brite adipose tissue, cold exposure also triggers the expansion of brown and beige/brite depots. Expansion and activation of brown and beige/brite adipose tissue is of great interest to treat obesity and diabetes due to its capacity to consume significant amounts of lipids and glucose. NE is currently believed to be the predominant neurotransmitter and effector of brown and beige/brite adipose expansion and activation. Unfortunately, NE is not useful for treatment for treatment of obesity and diabetes because it leads to increased blood pressure and heart rate.

There are currently no activators of brown and beige/brite adipose tissue for the treatment of obesity and/or diabetes. Among adrenergic activators, Mirabegron (Myrbetriq, Astellas Pharma, Inc.), a drug approved to treat overactive bladder, has been shown in proof-of-concept studies to activate human brown adipose (Cypess et al., 2015). However, this drug resulted in elevated blood pressure and heart rate, which are undesirable side effects for potential obese patients and lessen enthusiasm for this therapeutic avenue.

There is therefore a need for means that offer a more specific activation of brown and beige/brite adipose depots without the need for systemic administration of an adrenergic activator.

### SUMMARY OF INVENTION

The present disclosure relates to an agonist of Tacr2 or a pharmaceutically acceptable salt thereof for use in treatment of diabetes and/or obesity in an individual in need thereof. The inventors have found that agonists of Tacr2, in particular peptide agonists of Tacr2 are able of activating brown and beige/brite adipose tissue independently of norepinephrine (NE)/β-adrenergic receptor signalling. The invention discloses that administration of agonists of Tacr2 to an individual has a multiplicity of effects on brown and beige adipocytes, which are beneficial in treatment of obesity and diabetes. In particular, agonists of Tacr2 cause an increase in oxygen consumption rate, and preferably also in glucose absorption, oxygen consumption rate, energy consumption and heat production in brown and beige adipocytes. Preferably, agonists of Tacr2, in particular peptide agonists of Tacr2, result in activation of brown adipose tissue, without causing deleterious side effects such as elevated blood pressure and heart rate

One aspect of present disclosure relates to an agonist of Tacr2 or a pharmaceutically acceptable salt thereof, in particular a peptide agonist of Tacr2 for use in treatment of diabetes and/or obesity in an individual in need thereof.

Another aspect of the present disclosure relates to a pharmaceutical composition comprising a Tacr2 agonist or a pharmaceutically acceptable salt thereof, in particular the peptide agonist of Tacr2 as defined herein for treatment of obesity and/or diabetes in an individual in need thereof.

Another aspect of the present disclosure relates to the use of an agonist of Tacr2 or a pharmaceutically acceptable salt thereof, in particular a peptide agonist of Tacr2 for manufacture of a medicament for treatment of diabetes and/or obesity.

Another aspect of the present disclosure relates to a method for treatment of diabetes and/or obesity in an individual in need thereof, wherein the method comprises administering a therapeutically effective amount of an agonist of Tacr2 or a pharmaceutically acceptable salt thereof, in particular a peptide agonist of Tacr2 to said individual.

Another aspect of the present disclosure relates to a method for treatment of diabetes and/or obesity in an individual in need thereof, wherein the method comprises enhancing activity of brown adipose tissue cells in said individual by administration of a therapeutically effective amount of an agonist of Tacr2 or a pharmaceutically acceptable salt thereof, in particular a peptide agonist of Tacr2 to said individual.

### DESCRIPTION OF DRAWINGS

FIG. 1: Expressional and functional characterization of NKA/TACR2 in brown and white adipose tissue. Expression profile of Tacr2 in cold-induced (a) interscapular brown adipose tissue (iBAT), (b) inguinal white adipose tissue (iWAT) and (c) epididymal white adipose tissue (eWAT) relative to thermoneutrality from C57Bl/6 mice. Data shows means and SEMs of n=5.

FIG. 2: (a) Tacr2 mRNA expression levels were determined in mature adipocyte and stromal vascular fractions from iBAT of thermoneutrally acclimated or cold-challenged mice. (b) Tacr2 mRNA expression in iBAT from C57Bl/6 mice fed either chow diet or 60% high fat diet (HFD) for 12 weeks. Data shows means and SEMs of n=8. (c)

FIG. 3: (a) NKA-mediated IP3 production in primary brown adipocytes transfected with either Tacr2 or vector control. (b) Oxygen consumption rate (OCR), measured by Seahorse Bioscience Analyzer, in primary brown adipocytes, that have been treated with NKA or vehicle for 72 hours. Measurement of basal respiration was followed by NE (5 μM), oligomycin (1 μM), FCCP (1 μM) and rotenone and antimycin A (1 μM each). Data shows means and SEMs, n=8.

FIG. 4: (a) Mice were injected twice daily with 1 mg/kg NKA (grey) or vehicle (black), marked by arrows, during the light period for nine consecutive days. Oxygen consumption was measured every 5 minutes using the PhenoMaster. Data is representative of all days and show means±SEM of n=6. (b) Weight was measured every day throughout the experimental period. Data show means±SEM of n=6-7, *p<0.05, **p<0.01 using two-way ANOVA with Sidak's multiple comparisons. (c) Insulin tolerance test was performed in 3h fasted mice the day after treatment, and mice received 1 UI per kg lean body mass insulin intraperitoneal. Data show means±SEM of n=6-7, *p<0.05, **p<0.01 using two-way ANOVA with Sidak's multiple comparisons. (d) The three major adipose tissues were harvested weighed upon termination of experiment. Data show means±SEM of n=6-7, *p<0.05 using unpaired Student's t-test.

FIG. 5: Weight loss; b) food intake; and c) calorie burning in mice treated with either vehicle (black) or [Lys2- [symbol] -Glu-C₁6_{]}NKA (grey). Data shows means+/−SEM of n=6.

### DETAILED DESCRIPTION OF THE INVENTION

### Definitions

The term “agonist” as used herein relates to an agonist of Tacr2 unless otherwise specifically stated. An agonist of Tacr2 as defined herein refers to a compound comprising or consisting of a peptide that can bind Tacr2 thereby enhancing its activity.

The term “activity of brown and/or beige/brite adipose tissue cells” such as “activity of brown and/or beige/brite adipocytes” as used herein refers to a number of biochemical parameters typical of cells of the brown and beige/brite adipose tissue that can vary in response to the interaction between Tacr2 and an agonist. Some examples of parameters that define activity of brown and/or beige/brite adipocytes are basal oxygen consumption rate (OCR), norepinephrine (NE)-induced OCR, maximal respiration, glucose uptake/absorption, lipid uptake/absorption, heat production.

Proteinogenic “amino acids” are named herein using its 1-letter code according to the recommendations from IUPAC, see for example http://www.chem.qmw.ac.uk/iupac. If nothing else is specified an amino acid may be of D or L-form.

The term “ Cₘᵢₙ” as used herein refers to the lowest lowest blood concentration of a compound. Thus, Cₘᵢₙ in relation to blood glucose levels after administration of insulin is the lowest blood glucose concentration observed after said administration. Frequently, Cₘᵢₙ in relation to blood glucose levels is the lowest blood glucose concentration observed within an insulin dosing interval.

The term “functional analogue” as used herein refers to a compound that acts in a similar manner as a reference compound. Thus a functional analogue of a peptide agonist of Tacr2 acts as a peptide agonist of Tacr2. The functional analogue can for example be a compound comprising a peptide, wherein the peptide may be modified with moieties that do not necessarily consist of amino acid residues, that can bind Tacr2 thereby enhancing its activity.

“Non-standard amino acids” are amino acid residues that are not encoded by the genetic code of any organism. Non-standard amino acid residue may occur naturally in organisms. Non limiting examples of non-standard amino acids are: natural amino acids in D-conformation, β amino acid, γ amino acid and non-proteinogenic amino acids.

The term “PEG”, polyethylene glycol, refers to a polymer of ethylene glycol having chemical formula C₂ₙH₄ₙ₊₂Oₙ₊₁ and the repeating structure:

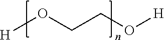

The term “peptide”, as used herein is a sequence of at least 2 amino acid residues linked via amide bonds.

The term “peptide agonist” refers to a compound agonist of Tacr2 unless otherwise specifically stated. A peptide agonist of Tacr2 as defined herein refers to compound comprising or consisting of a peptide that can bind Tacr2 thereby enhancing its activity.

The peptide agonist of Tacr2 may consist of a peptide and a conjugated moiety. The conjugated moiety may for example be a sugar, a lipid, a second peptide or any other chemical group that the skilled person would consider beneficial.

The term “sequence identity” as used herein refers to the % of identical amino acids or nucleotides between a candidate sequence and a reference sequence following alignment. Thus, a candidate sequence sharing 80% amino acid identity with a reference sequence requires that, following alignment, 80% of the amino acids in the candidate sequence are identical to the corresponding amino acids in the reference sequence. Identity according to the present invention is determined by aid of computer analysis, such as, without limitations, the Clustal Omega computer alignment program for alignment of polypeptide sequences (Sievers et al. (2011 Oct. 11) Molecular Systems Biology 7:539, PMID: 21988835; Li et al. (2015 Apr. 6) Nucleic Acids Research 43 (W1):W580-4 PMID: 25845596; McWilliam et al., (2013 May 13) Nucleic Acids Research 41 (Web Server issue):W597-600 PMID: 23671338), and the default parameters suggested therein. The Clustal Omega software is available from EMBL-EBI at https://www.ebi.ac.uk/Tools/msa/clustalo/. Using this program with its default settings, the mature (bioactive) part of a query and a reference polypeptide are aligned. The number of fully conserved residues are counted and divided by the length of the reference polypeptide. The MUSCLE or MAFFT algorithms may be used for alignment of nucleotide sequences. Sequence identities may be calculated in a similar way as indicated for amino acid sequences. Sequence identity as provided herein is thus calculated over the entire length of the reference sequence.

The term “treatment” as used herein refers to management and care of a patient for the purpose of combating a condition, disease or disorder. The term is intended to include the full spectrum of treatments for a given condition from which the patient is suffering, such as administration of the active compound for the purpose of: alleviating or relieving symptoms or complications; delaying the progression of the condition, disease or disorder; ameliorating, curing or eliminating the condition, disease or disorder; and/or preventing the condition, disease or disorder. The patient to be treated is preferably a mammalian, in particular a human being. The patients to be treated can be of various ages.

### Agonist of Tacr2

One aspect of the present disclosure relates to an agonist of Tacr2 or pharmaceutically acceptable salts thereof, in particular a peptide agonist of Tacr2 for use in treatment of obesity and/or diabetes. Agonists of Tacr2 may be any compound that can bind Tacr2 and enhance its activity. In some embodiments, an agonist of Tacr2 is a peptide agonist. Said peptide agonist of Tacr2 may comprise or consist of a peptide and optionally a conjugated moiety, as described in detail in the sections below “Peptide agonist” and “Conjugated moiety”.

Preferably, agonists of Tacr2 are capable of enhancing the activity of Tacr2 as determined by any suitable assay, in particular by a cellular functional assays that can measure the activity of Tacr2, for example the Ca²⁺-mobilization assay or the production of inositol-3-phosphate (IP3), both described by Ablas J. et al. (1995). Radioligand assays or other assays, for example functional ‘monoreceptorial’ bioassays can be chosen by a skilled person as suitable means for measuring Tacr2 activity. Thus, the agonist of Tacr2 may be any compound, such as an peptide agonist, which is capable of enhancing the activity of Tacr2 as determined by any of the aforementioned assays, preferably capable of enhancing the activity of Tacr2 by at least 20%, such as by at least 30%, for example by at least 40%.

In some embodiments, the agonist activity is measured by determining IP3 production in a cell expressing Tacr2. Preferably, an agonist of Tacr2 is a compound, which upon contact with such cells results in increased production of IP3. Thus, it may be determined whether a compound is an agonist of Tacr2 by a method involving the steps of
Providing a cell expressing Tacr2
Contacting said cells with myo-inositol and with a compound, which is a putative agonist of Tacr2
Determining the production of IP3 in said cells in the absence and presence of said putative agonist of Tacr2

wherein increased production of IP3 in the presence of said compound is indicative of said compound being an agonist of Tacr2.

The cells may express Tacr2 endogenously or they may be cells recombinantly manipulated to express Tacr2, e.g the cells may be COS-7 or HEK 293 cells comprising a heterologous nucleic acid encoding Tacr2. Production of IP3 may be determined as a dose-response curve.

The cells may also be primary brown adipocytes, e.g. primary brown adipocytes comprising a heterologous nucleic acid encoding Tacr2. Thus, it may be preferred that the agonist of Tacr2 is a compound that induces production of IP3 in an assay performed as described in Example 2. Preferably, said agonist is a compound that induces production of IP3 in a similar manner as NKA or induces production of at least 80% IP3 as compared to NKA, when determined as described in Example 2.

The agonist of Tacr2 may therefore be a compound that enhances the activity of Tacr2 by at least 20% as measured using an assay that monitors IP3 production as described above. The agonist of Tacr2 may also be a compound that enhances the activity of Tacr2 by at least 30% as measured using an assay that monitors IP3 production as described above.

The agonist of Tacr2 may also be a compound that enhances the activity of Tacr2 by at least 40% as measured using an assay that monitors IP3 production as described above.

Agonists of Tacr2 preferably have high affinity for Tacr2. The affinity of a compound for a receptor, i.e. the affinity of a compound for Tacr2, can be measured via different assays and so results in different affinity units, as indicated in Table 1, which are described below:
pIC₅₀ is the negative logarithm to base 10 of the IC₅₀ of an agonist, where IC₅₀ is a term used in a number of ways, in particular it may be the molar concentration of an unlabeled agonist that inhibits the binding of a radioligand by 50%; the concentration of radioligand should be given; the unit of IC₅₀ in the present disclosure is M (molar) unless specified otherwise;
pEC₅₀ is the negative logarithm to base 10 of the EC₅₀ of an agonist, where EC₅₀, is the molar concentration of an agonist that produces 50% of the maximal possible effect, measured with an appropriate assay as defined above in this section, of that agonist. Other percentage values (EC₂₀, EC₄₀, etc.) can be specified; the unit of EC₅₀ in the present disclosure is M (molar) unless specified otherwise;
pK_{d} is the negative logarithm to base 10 of the K_{d}, where K_{d} refers to the equilibrium dissociation constant of an agonist determined directly in a binding assay using a labeled form of the agonist;
pKᵢ is the negative logarithm to base 10 of the Kᵢ, where Kᵢ refers to the equilibrium dissociation constant of a ligand determined in inhibition studies. The Kᵢ for a given ligand is typically (but not necessarily) determined in a competitive radioligand binding study by measuring the inhibition of the binding of a reference radioligand by the competing ligand of interest under equilibrium conditions

In particular, the agonist of Tacr2, e.g. the peptide agonist may be a compound having a pEC₅₀ of 8 to 10, as measured in a test such as radioligand binding assay or ‘monoreceptorial’ bioassay as described in Bellucci F. et al. (2002).

In some embodiments the peptide agonist may be a compound having a pEC₅₀ for the murine receptor in the range of 6.7 to 9.5 when determined as described in Example 7.

For example the peptide agonist may be a compound having a pEC₅₀ for the murine receptor of at least 6.8, such as a pEC₅₀ of at least 7, preferably a pEC₅₀ of at least 7.5, for example a pEC₅₀ of at least 8, such as a pEC₅₀ of at least 8.5, preferably a pEC₅₀ of at least 9.

In some embodiments the peptide agonist may be a compound having a pEC₅₀ for the human receptor in the range of 7.2 to 9.5 when determined as described in Example 7.

For example the peptide agonist may be a compound having a pEC₅₀ for the human receptor of at least 7.5, such as a pEC₅₀ of at least 7.8, preferably a pEC₅₀ of at least 8, for example a pEC₅₀ of at least 8.5, such as a pEC₅₀ of at least 9,

In particular, the agonist of Tacr2, e.g. the peptide agonist may be a compound having a pK_{d} of 6 to 10, as measured in in a test such as radioligand binding assay or Thonoreceptoriali bioassay as described in Bellucci F. et al. (2002).

In particular, the agonist of Tacr2, e.g. the peptide agonist may be a compound having a pKᵢ of 5 to 10, as measured in in a test such as radioligand binding assay or ‘monoreceptorial’ bioassay as described in Bellucci F. et al. (2002).

It may also be determined whether a potential agonist has high affinity for Tacr2 using an assay that measure the activity of Tacr2 as described in the beginning of this section. The agonists are those compounds, preferably peptides agonists, that result in increased Tacr2 activity, such as increased Ca²⁺-mobilization or inositol accumulation. Preferably, the peptides agonists of the present disclosure are also characterized by having higher affinity for the Tacr2 than for the Tacr1. They may have low or negligible affinity for Tacr1. They may also be able to bind Tacr1, but their affinity for Tacr2 is higher than that for Tacr1. Thus, preferably agonists of Tacr2, such as peptide agonists of Tacr2 have an affinity for the Tacr2 which is at least 2×, preferably at least 5× higher than the affinity for Tacr1.

Agonists of Tacr2 can be both naturally occurring and artificial compounds. A list of non-limiting examples of natural agonists of Tacr2 found in human (Homo sapiens, Hs), rat (Rattus norvegicus, Rn) and guinea pig (Cavia porcellus, Cp) and their affinity to Tacr2 are listed in Table 1 (Douglas S. D., et al. 2015).

TABLE 1
Agonists of Tacr2 and their affinity for the receptor.

| Ligand | Sp | Affinity | Units | Reference |
|---|---|---|---|---|
| neuropeptide γ {Sp: | Cp | 9.5 | pEC₅₀ | van Giersbergen P L et al. 1992 |
| Human, Mouse, | | | | |
| Rat} | | | | |
| neurokinin A {Sp: | Cp | 8.9 | pEC₅₀ | D'Orléans-Juste P, et al. 1986 |
| Human, Mouse, | | | | |
| Rat} | | | | |
| neuropeptide K {Sp: | Cp | 8.8 | pEC₅₀ | van Giersbergen P L et al. 1992 |
| Human, Rat} | | | | |
| [125I]NKA (human, | Hs | 9.3 | pK_{d} | Warner F J, et al. 1999 |
| mouse, rat) | | | | |
| [βAla8]neurokinin A- | Hs | 6.0 | pK_{d} | Emonds-Alt X, et al. 1993 |
| (4-10) | | | | |
| neurokinin A {Sp: | Hs | 8.0-9.1 | pKᵢ | Bellucci F. et al. 2002, Emonds-Alt |
| Human, Mouse, | | | | X, et al. 1993, Brawner M E et al. |
| Rat} | | | | 1997, Warner F J et al. 2001 |
| [Phe(Me)7]neurokinin B | Hs | 6.9 | pKᵢ | Brawner M E et al. 1997 |
| substance P {Sp: | Hs | 5.9-6.9 | pKᵢ | Bellucci F. et al. 2002, Emonds-Alt |
| Human, Mouse, | | | | X, et al. 1993, Brawner M E et al. |
| Rat} | | | | 1997 |
| neurokinin B {Sp: | Hs | 5.0-7.7 | pKᵢ | Emonds-Alt X, et al. 1993, |
| Human, Mouse, | | | | Brawner M E et al. 1997 |
| Rat, Pig} | | | | |
| hemokinin 1 {Sp: | Hs | 6.3 | pKᵢ | Bellucci F. et al. 2002 |
| Mouse} | | | | |
| GR64349 | Rn | 8.4 | pEC₅₀ | Deal M J et al. 1992 |
| [Lys5,Me- | Rn | 8.8-9.4 | pIC₅₀ | Matuszek M A et al. 1998 |
| Leu9,Nle10]NKA-(4- | | | | |
| 10) | | | | |

The agonist of Tacr2, e.g. the peptide agonist may be a compound having high efficacy, which can be for example measured in a test such as radioligand binding assay or ‘monoreceptorial’ bioassay as described in Bellucci F. et al. (2002), where the top value indicates the efficacy of the tested agonist and the difference between the top and the bottom value indicate the efficacy span of the tested value.

In one embodiment of the present disclosure, the agonist of Tacr2, e.g. the peptide agonist may be a compound having as high efficacy as possible. The efficacy may for example be obtained from the top value obtained in test such as radioligand binding assay or Thonoreceptoriali bioassay as described in Bellucci F. et al. (2002).

In one embodiment of the present disclosure, the agonist of Tacr2, e.g. the peptide agonist may be a compound having an efficacy for the murine receptor of at least 1260 relative units, such as of at least 1300 relative units, such as of at least 1400 relative units, such as of at least 1500 relative units, such as of at least 1600 relative units, such as of at least 1700 relative units, such as of at least 1800 relative units, such as of at least 1900 relative units, such as of at least 2000 relative units, such as of at least 2400 relative units, such as of at least 2700 relative units, when determined as described in Example 7.

In one embodiment of the present disclosure, the agonist of Tacr2, e.g. the peptide agonist may be a compound having an efficacy for the human receptor of at least 2000 relative units, such as of at least 2200 relative units, such as of at least 2400 relative units, such as of at least 2600 relative units, such as of at least 2800 relative units, such as of at least 3000 relative units, when determined as described in Example 7.

In one embodiment of the present disclosure, the agonist of Tacr2, e.g. the peptide agonist may be a compound having as large efficacy span as possible. The efficacy span may for example be measured by subtracting the bottom value from the top value obtained in test such as radioligand binding assay or ‘monoreceptorial’ bioassay as described in Bellucci F. et al. (2002).

In one embodiment of the present disclosure, the agonist of Tacr2, e.g. the peptide agonist may be a compound having an efficacy span in relation to the murine receptor of at least 1100 relative units, such as of at least 1300 relative units, such as of at least 1400 relative units, such as of at least 1500 relative units, such as of at least 1600 relative units, such as of at least 1700 relative units, such as of at least 1800 relative units, such as of at least 1900 relative units, such as of at least 2000 relative units, such as of at least 2400 relative units, such as of at least 2700 relative units, when determined as described in Example 7.

In one embodiment of the present disclosure, the agonist of Tacr2, e.g. the peptide agonist may be a compound having an efficacy span in relation to the human receptor of at least 1700 relative units, such as of at least 2000 relative units, such as of at least 2200 relative units, such as of at least 2400 relative units, such as of at least 2600 relative units, such as of at least 2800 relative units, such as of at least 3000 relative units, when determined as described in Example 7.

### Peptide Agonist

One aspect of the present disclosure relates to a peptide agonist of Tacr2 or a pharmaceutically acceptable salt thereof for use in treatment of diabetes and/or obesity in an individual in need thereof, where the peptide agonist comprises or consists of a peptide. When the peptide agonist of Tacr2 comprises a peptide, said peptide may be covalently linked to a conjugated moiety, as described in the section below “Conjugated moiety”. Preferably, the peptide agonist is able to bind Tacr2 and to cause an increase of Ca²⁺-mobilization or inositol accumulation in cells expressing Tacr2. The peptide agonist is characterized by having high affinity for Tacr2 as described in the section above “Agonist of Tacr2”. Some examples of peptide agonists of Tacr2 and their affinity are listed in Table 1.

In one embodiment, the peptide agonist comprises or consists of Neurokinin A or a functional analogue thereof. Neurokinin A, also known as Substance K, is a peptide translated from the pre-protachykinin gene. Neurokinin A can be translated via alternative splicing from different isoforms of the pre-protachykinin gene. In humans, there are two isoforms of the gene encoding for Neurokinin A and so two precursor proteins, protachykinin-1 isoform beta precursor (SEQ ID NO:12) and protachykinin-1 isoform gamma precursor (SEQ ID NO:13). Similarly, two isoforms of the gene encoding for Neurokinin A, and so two precursor protein, exist also in mouse and are named protachykinin-1 isoform 2 precursor (SEQ ID NO:14) and protachykinin-1 isoform 1 precursor (SEQ ID NO:15).

In some embodiments, the peptide agonist of Tacr2 comprises or consists of a precursor protein of Neurokinin A or a fragment thereof. Accordingly, in some embodiments, the peptide agonist comprises the precursor protein of Neurokinin A of SEQ ID NO:12, SEQ ID NO:13, SEQ ID NO:14 or SEQ ID NO:15 or a functional analogue thereof sharing at least 75%, such as at least 80%, for example at least 85%, such as at least 90%, for example at least 95% sequence identity therewith. Preferably, the peptide agonist comprises or consists of human protachykinin-1 isoform beta precursor or a fragment thereof. More preferably, the peptide agonist comprises or consists of human protachykinin-1 isoform gamma precursor or a fragment thereof. More preferably, the peptide agonist comprises or consists of murine protachykinin-1 isoform 2 precursor or a fragment thereof. More preferably, the peptide agonist comprises or consists of murine protachykinin-1 isoform 1 precursor or a fragment thereof.

In some embodiments the peptide agonist comprise a fragment of the precursor protein of Neurokinin A of SEQ ID NO:12, SEQ ID NO:13, SEQ ID NO:14 or SEQ ID NO:15 or a functional analogue thereof sharing at least 75%, such as at least 80%, for example at least 85%, such as at least 90%, for example at least 95% sequence identity therewith. Accordingly, in some embodiments, the peptide agonist consists of a peptide optionally linked to a conjugated moiety, wherein the peptide consists of a consecutive sequence of at most 130 amino acid residues of SEQ ID NO:12, SEQ ID NO:13, SEQ ID NO:14 or SEQ ID NO:15, or of a functional analogue of any of the aforementioned sharing at least 75%, such as at least 80%, for example at least 85%, such as at least 90%, for example at least 95% sequence identity therewith. For example, in some embodiments, the peptide agonist may consist of a peptide optionally linked to a conjugated moiety, wherein the peptide consists of a consecutive sequence of at most 130 amino acid residues of SEQ ID NO:12, SEQ ID NO:13, SEQ ID NO:14 or SEQ ID NO:15, or of a functional analogue of any of the aforementioned sharing at least 75%, such as at least 80%, for example at least 85%, such as at least 90%, for example at least 95% sequence identity therewith, wherein said peptide comprises the sequence of SEQ ID NO:16 and/or of SEQ ID NO:30.

Preferably, the peptide agonist of Tacr2 is a functional analogue of Neurokinin A. In some embodiments, the peptide agonist of Tacr2 comprises or consists of the consensus sequence X₆X₁FX₂X₃X₄X₅ [SEQ ID NO:30] and wherein
X₆=aspartic acid (E) or glutamic acid (D);
X₁=serine (S) or lysine (K);
X₂=valine (V) or tryptophan (W);
X₃=glycine (G), β-alanine (β-ala);
X₄=methyl-leucine (Me-Leu), L, γ-lactam-leucine (γ-lactam-Leu);
X₅=norleucine (Nle), methionine (M).

Even more preferably, the peptide agonist of Tacr2 consists of a peptide optionally linked to a conjugated moiety, wherein the peptide consists of a sequence of at least 7 amino acids and at most 130 amino acids, wherein the sequence comprises SEQ ID NO:30.

In another embodiment, the peptide agonist of Tacr2 consists of a peptide optionally linked to a conjugated moiety, wherein the peptide consists of at least 7 and at most 130 amino acid residues comprising the sequence of SEQ ID NO: 30. Preferably, the peptide consists of at least 7 and at most 100 amino acid residues comprising the sequence of SEQ ID NO: 30, such as at least 7 and at most 70 amino acid residues comprising the sequence of SEQ ID NO: 30, such as at least 7 and at most 50 amino acid residues comprising the sequence of SEQ ID NO: 30, such as at least 7 and at most 30 amino acid residues comprising the sequence of SEQ ID NO: 30, such as at least 7 and at most 20 amino acid residues comprising the sequence of SEQ ID NO: 30, such as at least 7 and at most 15 amino acid residues comprising the sequence of SEQ ID NO: 30. In a further embodiment, the peptide agonist consists of 7 amino acid residues of SEQ ID NO:30.

Preferably, the peptide agonist of Tacr2 is a functional analogue of Neurokinin A. In some embodiments, the peptide agonist of Tacr2 comprises or consist of the consensus sequence DX₁FX₂X₃X₄X₅ [SEQ ID NO:16] and wherein
X₁=serine (S) or lysine (K);
X₂=valine (V) or tryptophan (W);
X₃=glycine (G), β-alanine (β-ala);
X₄=methyl-leucine (Me-Leu), L, γ-lactam-leucine (γ-lactam-Leu);
X₅=norleucine (Nle), methionine (M).

Even more preferably, the peptide agonist of Tacr2 consists of a peptide optionally linked to a conjugated moiety, wherein the peptide consists of a sequence of at least 7 amino acids and at most 130 amino acids, wherein the sequence comprises SEQ ID NO:16.

In some embodiments, the peptide agonist may comprise non-standard amino acid residues such as D-γ-lactam, β-alanine, γ-lactam-leucine, methyl-leucine, norleucine or other amino acids that may improve the affinity of the peptide agonist for Tacr2 or its agonistic behaviour.

In another embodiment, the peptide agonist of Tacr2 consists of a peptide optionally linked to a conjugated moiety, wherein the peptide consists of at least 7 and at most 130 amino acid residues comprising the sequence of SEQ ID NO:16. Preferably, the peptide consists of at least 7 and at most 100 amino acid residues comprising the sequence of SEQ ID NO:16, such as at least 7 and at most 70 amino acid residues comprising the sequence of SEQ ID NO:16, such as at least 7 and at most 50 amino acid residues comprising the sequence of SEQ ID NO:16, such as at least 7 and at most 30 amino acid residues comprising the sequence of SEQ ID NO:16, such as at least 7 and at most 20 amino acid residues comprising the sequence of SEQ ID NO:16, such as at least 7 and at most 15 amino acid residues comprising the sequence of SEQ ID NO:16. In a further embodiment, the peptide agonist consists of 7 amino acid residues of SEQ ID NO:16.

In another embodiment, the peptide agonist of Tacr2 comprises or consists of a sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:21, SEQ ID NO:22, SEQ ID NO:23, SEQ ID NO:24, SEQ ID NO:25, SEQ ID NO:26, SEQ ID NO:27 and any of the aforementioned wherein at the most 2 amino acids have been exchanged with a standard or non-standard amino acid.

In particular, the peptide agonist of Tacr2 may comprises or consists of a sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:21, SEQ ID NO:22, SEQ ID NO:23, SEQ ID NO:24, SEQ ID NO:25, SEQ ID NO:26, SEQ ID NO:27 and any of the aforementioned wherein at the most 1 amino acids have been exchanged. More preferably, the peptide agonist of Tacr2 comprises or consists of a sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8,SEQ ID NO:9, SEQ ID NO:21, SEQ ID NO:22, SEQ ID NO:23, SEQ ID NO:24, SEQ ID NO:25, SEQ ID NO:26 and SEQ ID NO:27.

In another embodiment, the peptide agonist of Tacr2 consists of a peptide optionally linked to a conjugated moiety, wherein the peptide consists of at least 7 and at most 130 amino acid residues comprising a sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:21, SEQ ID NO:22, SEQ ID NO:23, SEQ ID NO:24, SEQ ID NO:25, SEQ ID NO:26, SEQ ID NO:27 and any of the aforementioned wherein at the most 2, such as at the most 1 amino acid has been exchanged with a standard or non-standard amino acid. In particular, the peptide agonist of Tacr2 may consist of a peptide optionally linked to a conjugated moiety, wherein the peptide consists of at least 7 and at most 100 amino acid residues comprising a sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:21, SEQ ID NO:22, SEQ ID NO:23, SEQ ID NO:24, SEQ ID NO:25, SEQ ID NO:26 and SEQ ID NO:27 such as of at least 7 and at most 70 amino acid residues comprising a sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8 SEQ ID NO:9, SEQ ID NO:21, SEQ ID NO:22, SEQ ID NO:23, SEQ ID NO:24, SEQ ID NO:25, SEQ ID NO:26 and SEQ ID NO:27 such as of at least 7 and at most 50 amino acid residues comprising a sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8,SEQ ID NO:9, SEQ ID NO:21, SEQ ID NO:22, SEQ ID NO:23, SEQ ID NO:24, SEQ ID NO:25, SEQ ID NO:26 and SEQ ID NO:27 such as of at least 7 and at most 30 amino acid residues comprising a sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8,SEQ ID NO:9, SEQ ID NO:21, SEQ ID NO:22, SEQ ID NO:23, SEQ ID NO:24, SEQ ID NO:25, SEQ ID NO:26 and SEQ ID NO:27 such as of at least 7 and at most 15 amino acid residues comprising a sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:21, SEQ ID NO:22, SEQ ID NO:23, SEQ ID NO:24, SEQ ID NO:25, SEQ ID NO:26 and SEQ ID NO:27.

Accordingly, in some embodiments, the peptide agonist consists of a peptide optionally linked to a conjugated moiety, wherein the peptide consists of a consecutive sequence of in the range of 7 to 130 amino acids, such as in the range of 7 to 100, for example in the range of 7 to 50, such as in the range of 7 to 15 consecutive amino acids of SEQ ID NO:12, SEQ ID NO:13, SEQ ID NO:14 or SEQ ID NO:15, or a fragment of any of the aforementioned, or of a functional analogue of any of the aforementioned sharing at least 75%, such as at least 80%, for example at least 85%, such as at least 90%, for example at least 95% sequence identity therewith, wherein the peptide comprises a sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8,SEQ ID NO:9, SEQ ID NO:21, SEQ ID NO:22, SEQ ID NO:23, SEQ ID NO:24, SEQ ID NO:25, SEQ ID NO:26 and SEQ ID NO:27.

In one preferred embodiment, the peptide agonist consists of a peptide optionally linked to a conjugated moiety, wherein the peptide consists of a sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:21, SEQ ID NO:22, SEQ ID NO:23, SEQ ID NO:24, SEQ ID NO:25, SEQ ID NO:26, SEQ ID NO:27.

In another embodiment, the peptide agonist of Tacr2 consists of a peptide optionally linked to a conjugated moiety, wherein the peptide consists of at least 7 and at most 130 amino acid residues comprising the sequence SEQ ID NO:2, wherein at the most 2, such as at the most 1 amino acid has been exchanged with a standard or non-standard amino acid. In particular, the peptide agonist of Tacr2 may consist of a peptide optionally linked to a conjugated moiety, wherein the peptide consists of at least 7 and at most 100 amino acid residues comprising the sequence SEQ ID NO:2, such as at least 7 and at most 70 amino acid residues comprising the sequence SEQ ID NO:2, such as at least 7 and at most 50 amino acid residues comprising the sequence SEQ ID NO:2, such as at least 7 and at most 30 amino acid residues comprising the sequence SEQ ID NO:2, such as at least 7 and at most 15 amino acid residues comprising the sequence SEQ ID NO:2.

Accordingly, in some embodiments, the peptide agonist consists of a peptide optionally linked to a conjugated moiety, wherein the peptide consists of a consecutive sequence of in the range of 7 to 130 amino acids, such as in the range 7 to 100, for example in the range of 7 to 50, such as in the range of 7 to 15 consecutive amino acids of SEQ ID NO:2, wherein at the most 2, such as at the most 1 amino acid has been exchanged with a standard or non-standard amino acid.

In one preferred embodiment, the peptide agonist consists of a peptide optionally linked to a conjugated moiety, wherein the peptide consists of the sequence SEQ ID NO:2.

In another embodiment, the peptide agonist of Tacr2 consists of a peptide optionally linked to a conjugated moiety, wherein the peptide consists of at least 10 and at most 130 amino acid residues comprising the sequence SEQ ID NO:1, wherein at the most 2, such as at the most 1 amino acid has been exchanged with a standard or non-standard amino acid. In particular, the peptide agonist of Tacr2 may consist of a peptide optionally linked to a conjugated moiety, wherein the peptide consists of at least 10 and at most 100 amino acid residues comprising the sequence SEQ ID NO:1, such as at least 10 and at most 70 amino acid residues comprising the sequence SEQ ID NO:1, such as at least 10 and at most 50 amino acid residues comprising the sequence SEQ ID NO:1, such as at least 10 and at most 30 amino acid residues comprising the sequence SEQ ID NO:1, such as at least 10 and at most 15 amino acid residues comprising the sequence SEQ ID NO:1.

Accordingly, in some embodiments, the peptide agonist consists of a peptide optionally linked to a conjugated moiety, wherein the peptide consists of a consecutive sequence of in the range of 10 to 130 amino acids, such as in the range 10 to 100, for example in the range of 10 to 50, such as in the range of 10 to 15 consecutive amino acids of SEQ ID NO:1, wherein at the most 2, such as at the most 1 amino acid has been exchanged with a standard or non-standard amino acid.

In one preferred embodiment, the peptide agonist consists of a peptide optionally linked to a conjugated moiety, wherein the peptide consists of the sequence SEQ ID NO:1, wherein at the most 2, such as at the most 1 amino acid has been exchanged. In one preferred embodiment, the peptide agonist consists of a peptide optionally linked to a conjugated moiety, wherein the peptide consists of the sequence SEQ ID NO:1.

In some embodiments, the peptide agonist comprises or consist of the sequence DSFVGLM (SEQ ID NO:2). Preferably, the peptide agonist comprises or consist of Neurokinin A (SEQ ID NO:1). Preferably, the peptide agonist is Neurokinin A (SEQ ID NO:1).

In another embodiment, the peptide agonist of Tacr2 consists of a peptide optionally linked to a conjugated moiety, wherein the peptide consists of at least 10 and at most 130 amino acid residues comprising a sequence selected from the group consisting of SEQ ID NO:21, SEQ ID NO:22, SEQ ID NO:23, SEQ ID NO:24, SEQ ID NO:25, SEQ ID NO:26 and SEQ ID NO:27, wherein at the most 2, such as at the most 1 amino acid has been exchanged with a standard or non-standard amino acid. In particular, the peptide agonist of Tacr2 may consist of a peptide optionally linked to a conjugated moiety, wherein the peptide consists of at least 10 and at most 100 amino acid residues comprising a sequence selected from the group consisting of SEQ ID NO:21, SEQ ID NO:22, SEQ ID NO:23, SEQ ID NO:24, SEQ ID NO:25, SEQ ID NO:26 and SEQ ID NO:27,such as at least 10 and at most 70 amino acid residues comprising a sequence selected from the group consisting of SEQ ID NO:21, SEQ ID NO:22, SEQ ID NO:23, SEQ ID NO:24, SEQ ID NO:25, SEQ ID NO:26 and SEQ ID NO:27, such as at least 10 and at most 50 amino acid residues comprising a sequence selected from the group consisting of SEQ ID NO:21, SEQ ID NO:22, SEQ ID NO:23, SEQ ID NO:24, SEQ ID NO:25, SEQ ID NO:26 and SEQ ID NO:27, such as at least 10 and at most 30 amino acid residues comprising a sequence selected from the group consisting of SEQ ID NO:21, SEQ ID NO:22, SEQ ID NO:23, SEQ ID NO:24, SEQ ID NO:25, SEQ ID NO:26 and SEQ ID NO:27, such as at least 10 and at most 15 amino acid residues comprising a sequence selected from the group consisting of SEQ ID NO:21, SEQ ID NO:22, SEQ ID NO:23, SEQ ID NO:24, SEQ ID NO:25, SEQ ID NO:26 and SEQ ID NO:27.

Accordingly, in some embodiments, the peptide agonist consists of a peptide optionally linked to a conjugated moiety, wherein the peptide consists of a consecutive sequence of in the range of 10 to 130 amino acids, such as in the range 10 to 100, for example in the range of 10 to 50, such as in the range of 10 to 15 consecutive amino acids of a sequence selected from the group consisting of SEQ ID NO:21, SEQ ID NO:22, SEQ ID NO:23, SEQ ID NO:24, SEQ ID NO:25, SEQ ID NO:26 and SEQ ID NO:27, wherein at the most 2, such as at the most 1 amino acid has been exchanged with a standard or non-standard amino acid.

In one preferred embodiment, the peptide agonist consists of a peptide optionally linked to a conjugated moiety, wherein the peptide consists of a sequence selected from the group consisting of SEQ ID NO:21, SEQ ID NO:22, SEQ ID NO:23, SEQ ID NO:24, SEQ ID NO:25, SEQ ID NO:26 and SEQ ID NO:27.

In some embodiments, the peptide agonist comprises or consist of a sequence selected from the group consisting of SKTDSFVGLM (SEQ ID NO:21), HKTDSFVGLX (SEQ ID NO:22), HKTESFVGLM (SEQ ID NO:23), HKTESFVGLX (SEQ ID NO:24), KTDSFVGLM (SEQ ID NO:25), KDSFVGLM (SEQ ID NO: 26), KESFVGLM (SEQ ID NO: 27).

### Conjugated Moiety

The agonist of Tacr2 may comprise or consist of a peptide covalently linked to a conjugated moiety. For example, the peptide agonist may be any of the peptide agonists described in the section “Peptide agonist” herein above, wherein the conjugated moiety may be any of the conjugated moieties described in this section.

In one embodiment the conjugated moiety may be a peptide, a sugar, a lipid or any other chemical group that can be covalently linked to a peptide. Preferably, the conjugated moiety improves the agonistic behaviour of the agonist towards Tacr2. The conjugated moiety may also improve physical properties of the agonist of Tacr2, such as its solubility, stability or half-life.

In one embodiment, the conjugated moiety may be a compound that masks the agonist from the host immune system, such as a polyethylene glycol (PEG) polymer chain or a modified PEG, for example NPEG.

In some embodiments, a PEG moiety comprising between 1 and 50 ethylene glycol units is conjugated to the peptide agonist. The PEG moiety may comprise at least 2 ethylene glycol units, such as at least 4 ethylene glycol units, such as at least 6 ethylene glycol units. The PEG moiety can reduce the host immune reaction to the presence of the peptide agonist or it can increase its hydrodynamic size and so its circulatory time.

In some embodiments, the conjugated moiety facilitates interaction of the agonist with membranes and other biological structures. For example, one or more lipids, such as one or more fatty acids may be conjugated to the peptide agonist. The conjugated fatty acid may enhance hydrophobicity of the peptide agonist and therefore enhance its interaction with membranes. Examples of lipids that may be conjugated to the peptide agonist are palmitoleoyl group, prenyl groups, myristoyl group. Other lipid groups may also be conjugated.

In some embodiments, the conjugated moiety is a peptide, for example a peptide that facilitates cell penetration, such as a poly-arginine. Preferably, the conjugated moiety is a peptide that facilitates interaction of the agonist of Tacr2 with proteins and/or peptides present in a biological system. When advantageous, the conjugated moiety may be a hormone fragment.

In some embodiments, the agonist may be glycosylated, for example N-glycosylated or O-glycosylated. Glycosylation of the agonist may facilitate correct folding or facilitate recognition of other carbohydrate moieties present in a biological system, for example other glycosylated moieties. Some examples of glycans that can be conjugated to a peptide or be comprised in the agonist of Tacr2 are glycans comprising N-acetyl galactosamine, galactose, neuraminic acid, N-acetylglucosamine, fructose, mannose, and other monosaccharides.

In some embodiments the conjugated moiety is a saccharide, for example a monosaccharide, or a disaccharide, or a polysaccharide.

In some embodiments, the conjugated moiety is a saccharide, and said saccharide is conjugated to an oxygen atom in an amino acid residue of a peptide. For example, said saccharide may be conjugated to the hydroxyl group of a Serine.

In some embodiments, the conjugated moiety is a saccharide, and said saccharide is conjugated to a nitrogen atom in an amino acid residue of a peptide. For example, said saccharide may be conjugated to an amino group of Arginine.

In some embodiments, the conjugated moiety is a saccharide, for example a mannose (Man). For example, the conjugated peptide agonist comprises or consists of the sequence SEQ ID NO:21 and has a mannose conjugated to the Serine in position 12 of SEQ ID NO:21.

In some embodiments the conjugated moiety is an amine group. For example in embodiments of the invention where the agonist is a peptide agonist, said peptide may contain a C-terminal amidation, e.g. the C-terminal OH group of the peptide may be exchanged with an amine group.

In some embodiments the conjugated moiety is an acetyl group. For example in embodiments of the invention where the agonist is a peptide agonist, said peptide may contain an N-terminal acetylation, e.g. the N-terminal amine may be acetylated.

In some embodiments the conjugated moiety is attached to a terminal amino acid of the peptide agonist.

In some embodiments the conjugated moiety is attached to a terminal amino acid of the peptide agonist, wherein the peptide agonist comprises or consists of a sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:16, SEQ ID NO:21, SEQ ID NO:22, SEQ ID NO:23, SEQ ID NO:24, SEQ ID NO:25, SEQ ID NO:26, SEQ ID NO:27, and SEQ ID NO:30.

In some embodiments the conjugated moiety is attached to the amino group of a side chain of a non-terminal amino acid of the peptide agonist as defined herein, for example to the γ-N of a Lysine or of an Arginine.

In some embodiments the conjugated moiety is a lipid, and it is attached to the peptide agonist directly or via a further amino acid. In one embodiment the conjugated moiety is a fatty acid, for example the conjugated moiety may be C₆₋₄₀-alkylene-COOH, for example C₈₋₃₀-alkylene-COOH, for example C₁₀₋₂₅-alkylene-COOH. In one embodiment, the lipid is selected from the group consisting of capric acid, Lauric acid, Myristic acid, Palmitic acid, Stearic acid, and Arachidic acid.

In some embodiments the conjugated moiety is a fatty acid linked to an amino acid, for example the conjugated moiety may be -Naa-C₆₋₄₀-alkylene-COOH, for example -Naa-C₈₋₃₀-alkylene-COOH, for example -Naa-C₁₀₋₂₅-alkylene-COOH, wherein Naa may be any amino acid, for example any proteinogenic amino acid, such as Glu or Asp.

In some embodiments the conjugated moiety is -Naa-Myristic acid and it is attached to the γ-amino group of a non-terminal Lysine of the peptide agonist. For example, in one embodiment the conjugated peptide agonist is a peptide of SEQ ID NO: 1, wherein a Glutamate is connected to the [symbol] -N of the side chain of a Lysine in position 2 of SEQ ID NO: 1, and wherein Myristic acid is connected to the amino group of said Glutamate.

In some embodiments the conjugated moiety is -Naa-Palmitic acid and it is attached to the γ-amino group of a non-terminal Lysine of the peptide agonist. For example, in one embodiment the conjugated peptide agonist is a peptide of SEQ ID NO: 1, wherein a Glutamate is connected to the [symbol] -N of the side chain of a Lysine in position 2 of SEQ ID NO: 1, and wherein Palmitic acid is connected to the amino group of said Glutamate.

In some embodiments the conjugated moiety is -Naa-Stearic acid and it is attached to the γ-amino group of a non-terminal Lysine of the peptide agonist. For example, in one embodiment the conjugated peptide agonist is a peptide of SEQ ID NO: 1, wherein a Glutamate is connected to the [symbol] -N of the side chain of a Lysine in position 2 of SEQ ID NO: 1, and wherein Stearic acid is connected to the amino group of said Glutamate.

In some embodiments the conjugated moiety is Palmitic acid and it is attached to the amino group of the N-terminal amino acid of the peptide agonist. For example, in one embodiment the conjugated peptide agonist is a peptide of SEQ ID NO: 1, wherein Palmitic acid is connected to the amino group of the N-terminal amino acid of said peptide.

### Tacr2

The agonist is characterized by having high affinity for the Tachykinin receptor 2 (Tacr2) and for being an agonist of said receptor. Tacr2 is a G protein-coupled receptor (GPCR), which the inventors found to be induced in brown and beige/brite adipose tissue after cold exposure. The inventors have found that increasing the expression and presence of Tacr2 on the membrane of brown and beige/brite adipocytes, as well as increasing its activity by interaction of a peptide agonist with Tacr2, results in increased basal oxygen consumption rate (OCR), norepinephrine (NE)-induced OCR, and maximal respiration in brown and beige/brite adipocytes. Brown and beige/brite adipocytes consume fat and glucose upon activation with NE to produce heat.

The agonist of Tacr2 of the present invention can interact with the human Tacr2, which consists of SEQ ID NO:10 and also with murine Tacr2 which consists of SEQ ID NO:11.

### Pharmaceutical Composition

One aspect of the present disclosure relates to a pharmaceutical composition, and in particular to a pharmaceutical composition comprising the agonist of Tacr2 or a pharmaceutically acceptable salt thereof as defined herein for treatment of obesity and/or diabetes in an individual in need thereof.

The agonist as defined above in the section “Agonist of Tacr2” or a pharmaceutically acceptable salt thereof may be part of a pharmaceutical composition and so administered to an individual affected by obesity and/or diabetes, as described in the section below “Method for treatment of diabetes and/or obesity”.

Another aspect of the present disclosure relates to the use of the agonist of Tacr2 or a pharmaceutically acceptable salt thereof, as defined in the section above, for manufacture of a medicament for treatment of diabetes and/or obesity.

Whilst it is possible for the compounds or salts of the present invention to be administered as the raw peptide agonist, it is preferred to present them in the form of a pharmaceutical formulation. Accordingly, the present invention further provides a pharmaceutical formulation, which comprises a compound of the present invention or a pharmaceutically acceptable salt or ester thereof, as herein defined, and a pharmaceutically acceptable carrier therefor. The pharmaceutical formulations may be prepared by conventional techniques, e.g. as described in Remington (2005).

A pharmaceutical composition may comprise an agonist of Tacr2 or a pharmaceutically acceptable salt thereof as defined above and a pharmaceutically acceptable carrier and/or diluent. Pharmaceutically acceptable carriers and/or diluents are familiar to those skilled in the art. For compositions formulated as liquid solutions, acceptable carriers and/or diluents include saline and sterile water, and may optionally include antioxidants, buffers, bacteriostats, and other common additives.

The pharmaceutical compositions comprising the agonist of Tacr2 or a pharmaceutically acceptable salt thereof according to the invention may in particular be formulated to parenteral administration. Thus, the pharmaceutical composition of the present invention may be formulated in a wide variety of formulations for parenteral administration.

For injections and infusions the formulations may take such forms as suspensions, solutions, or emulsions in oily or aqueous vehicles, for example solutions in aqueous polyethylene glycol. Alternatively, the active ingredient may be in powder form, obtained by aseptic isolation of sterile solid or by lyophilisation from solution for constitution before use with a suitable vehicle, e.g., sterile, pyrogen-free water. The formulations can be presented in unit-dose or multi-dose sealed containers, such as ampoules, vials, pre-filled syringes, infusion bags, or can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets.

Examples of oily or non-aqueous carriers, diluents, solvents or vehicles include propylene glycol, polyethylene glycol, vegetable oils, and injectable organic esters, and may contain formulatory agents such as preserving, wetting, emulsifying or suspending, stabilizing and/or dispersing agents.

The pharmaceutical composition may be formulated for any kind of parenteral administration. In preferred embodiments, the pharmaceutical composition may be prepared for subcutaneous injection to an individual in need thereof.

### Method for Treatment of Insulin Resistance, Diabetes and/or Obesity

An aspect of the present disclosure relates to a method for treatment of insulin resistance, diabetes and/or obesity in an individual in need thereof, wherein the method comprises administering a therapeutically effective amount of an agonist of Tacr2, as defined in a section “Agonist of Tacr2”, to said individual. The invention also provides agonists of Tacr2 for use in such methods, e.g. for use in the methods described in this section.

Another aspect of the present disclosure relates to a method for treatment of insulin resistance, diabetes and/or obesity in an individual in need thereof, wherein the method comprises enhancing activity of brown adipose tissue cells in said individual by administration of a therapeutically effective amount of an agonist of Tacr2, as defined in a section “Agonist of Tacr2”, to said individual.

A further aspect of the present disclosure relates to a method of inducing weight loss in an individual in need thereof, wherein the method comprises administering a therapeutically effective amount of an agonist of Tacr2, as defined in a section “Agonist of Tacr2”, to said individual.

A further aspect of the present disclosure relates to a method of inducing weight loss in an individual in need thereof, wherein the method comprises enhancing activity of brown adipose tissue cells in said individual by administration of a therapeutically effective amount of an agonist of Tacr2, as defined in a section “Agonist of Tacr2”, to said individual.

In some embodiments, the agonist is administered in form of a pharmaceutical composition, as defined in the section “Pharmaceutical composition”. Frequently, the agonist is administered parenterally. For example, the agonist may be administered via injection. The agonist may be administered via a subcutaneous injection.

In some embodiments, the administered agonist binds Tacr2 in brown and beige adipocytes.

The administered agonist may have a multiplicity of effects on Tacr2. Thus, preferably it stimulates expression of Tacr2 and localization of Tacr2 on the cell membrane of brown and beige/brite adipocytes. It may preferably also increase glucose uptake, in particular glucose uptake by brown adipocytes. It may preferably also increase lipid uptake, in particular lipid uptake by brown adipocytes. It may preferably also increase energy consumption, in particular energy consumption by brown adipocytes. It may preferably also increase oxygen consumption, in particular oxygen consumption in brown adipocytes. It may preferably also increase heat production, in particular heat production in brown adipocytes. The effects are beneficial in treating obesity and/or diabetes.

In some embodiments, the method for treatment of diabetes and/or obesity in an individual in need thereof comprises increasing the basal oxygen consumption rate in primary brown adipocytes. Oxygen consumption in adipocytes may be measured using methods known in the art, for example measured using the Seahorse XF-96 Flux Analyzer, as also described in Example 2. The method for treatment of diabetes and/or obesity in an individual in need thereof may also comprise increasing norepinephrine-induced oxygen consumption rate in primary brown adipocytes. The method for treatment of diabetes and/or obesity in an individual in need thereof may also comprise increasing maximal oxygen consumption rate in primary brown adipocytes.

In some embodiments, the method for treatment of insulin resistance, diabetes and/or obesity in an individual in need thereof comprises increasing the absorption/uptake of glucose in brown and beige adipocytes. Accordingly, the method may result in reduced levels of blood glucose. Blood glucose levels may be assessed using methods known in the art. For example, Glucose Tolerance Test (GTT), also described in Example 4, or the oral glucose challenge test (OGCT) or other tests that the skilled person considers suitable.

In some embodiments, the method for treatment of insulin resistance, diabetes and/or obesity in an individual in need thereof comprises increasing the energy consumption in brown and beige adipocytes. Assays for measurement of the energy consumption of a cell are readily available in the art, for example, energy consumption can be measured by indirect calorimetry (i.e. O₂ consumption/CO₂ production) in live animals in metabolic cages, e.g. as described in Example 5. Energy dissipation can also be measured via thermometers implanted in an animal's tissue that record increases in the animal's core and brown adipose temperature in response to Neurokinin A.

In some embodiments, the method for treatment of insulin resistance, diabetes and/or obesity in an individual in need thereof comprises increasing the energy consumption in brown and beige adipocytes by administration of an agonist of Tacr2 as disclosed herein, without desensitization of said brown and beige adipocytes to said agonist of Tacr2.

In some embodiments, the method for treatment of insulin resistance, diabetes and/or obesity in an individual in need thereof comprises stimulating the release of endogenous neurokinin A in brown and beige adipocytes.

In some embodiments, the method for treatment of insulin resistance, diabetes and/or obesity in an individual in need thereof comprises increasing the expression of Tacr2 and consequently increasing its localization to the cell membrane of brown and beige/brite adipocytes.

In some embodiments, the method for treatment of diabetes and/or obesity in an individual in need thereof comprises inducing weight loss in said individual.

In some embodiments, said induced weight loss corresponds to a reduction of white adipose tissue.

In some embodiments, said induced weight loss does not correspond to a reduction of brown and beige/brite adipose tissue.

The dosage of the agonist of Tacr2 may be dependent on the particular agonist and the individual. In some embodiments, the agonist of Tacr2, in particular the agonist of

Tacr2 may for example be administered to a human individual at a dosage below 2 μg/kg body weight. In some embodiments, the agonist may for example be administered to a human individual at a dose between 0.05 and 2 μg/kg body weight, such as between 0.1 and 2 μg/kg body weight, such as 0.5 to 2 μg/kg body weight, such as 1 to 2 μg/kg body weight, such as between 0.1 and 1.5 μg/kg body weight, such as between 0.1 and 1 μg/kg body weight, such as between 0.1 and 0.5 μg/kg body weight.

In some embodiments, the individual in need thereof is affected by obesity and/or diabetes. The individual may be any individual, for example the individual may be a mammal, in particular a human being.

In one embodiment the invention relates to methods of treating diabetes, wherein said diabetes is for example selected from the group consisting of type 1 diabetes, type 2 diabetes and gestational diabetes. Said diabetes may in particular be diabetes associated with insulin resistance.

In some embodiments, the method for treatment of diabetes and/or obesity in an individual in need thereof comprises increasing the insulin sensitivity in said individual. Insulin sensitivity may be determined using methods known in the art, such as the Insulin Tolerance Test (ITT) described in Example 4 and 5.

In some embodiments, the invention provides a method for increasing insulin sensitivity and/or treatment of insulin resistance. Insulin resistance may be a precursor of diabetes 2 or it may be associated with diabetes 2. Thus, the method for treatment of diabetes and/or obesity may comprise increasing the insulin sensitivity in said individuals. Methods for determining insulin sensitivity are known in the art, and may for example be an Insulin Tolerance Test (ITT) where blood glucose levels are determined after administration of insulin. Useful ITT are described in Example 4 and 5, and the skilled person will be able to adapt the methods described therein to other mammals, e.g. to human beings. In one embodiment, insulin resistance is determined by determining the rate of whole-body glucose disposal (GDR) using a hyperinsulinemic-euglycemic clamp. GDR reflects the amount of exogenous glucose necessary to fully compensate for the hyperinsulinemia. The determination as to whether a particular individual suffers from insulin resistance may for example be performed as described in Tam et al., Diabetes Care. 2012 July; 35(7):1605-10. doi: 10.2337/dc11-2339.

Usually the blood glucose level decreases in consequence of administration of insulin and reaches a Cₘᵢₙ. After a certain period of time, the blood glucose level increases again typically to return to the levels registered before administration of insulin. Individuals suffering from insulin resistance respond to the administration of insulin in a different way and in particular their response is characterized by:
a Cₘᵢₙ higher than the Cₘᵢₙ of individuals that do not suffer from insulin resistance; and/or
a shorter time interval between reaching Cₘᵢₙ and returning “pre-insulin glucose levels”, which are the blood glucose levels registered before administration of insulin compared to individuals that do not suffer from insulin tolerance.

According to the methods of the present disclosure, an agonist of Tacr2 is administered for treatment of insulin resistance, diabetes and/or obesity in an individual in need thereof, for example to individuals suffering from or suspected of suffering from insulin resistance. Individuals suffering from or suspected of suffering from insulin resistance and treated according to the methods of the present disclosure, i.e. by administration of an agonist of Tacr2, respond to the administration of insulin in a different way compared to individuals suffering from or suspected of suffering from insulin resistance and not administered the agonist of Tacr2 as disclosed herein. In particular, individual which have received administration of an agonist of Tacr2 as described herein have a response characterized by:
lower Cₘᵢₙ; and/or
a longer time interval between reaching Cₘᵢₙ and returning to pre-insulin glucose levels.

Said response may be as compared to individuals that suffer from or are suspected of suffering from insulin resistance and are not administered the agonist of Tacr2 as disclosed herein or the same individual prior to treatment.

Therefore, in some embodiments, the method for treatment of diabetes and/or obesity in an individual in need thereof comprises reducing the blood glucose Cₘᵢₙ.

In some embodiments, the method for treatment of diabetes and/or obesity in an individual in need thereof comprises increasing the time interval between reaching the blood glucose Cₘᵢₙ in response to insulin and returning to pre-insulin glucose levels i.e. the blood glucose levels registered before administration of insulin, wherein said insulin is endogenous or exogenous.

In some embodiments, the present disclosure relates to a method for treatment of obesity and/or obesity-associated diseases in an individual in need thereof, wherein the method comprises administering a therapeutically effective amount of an agonist of Tacr2, as defined in a section “Agonist of Tacr2”, to said individual.

In other embodiments, the present disclosure relates to a method for treatment of obesity and obesity-associated diseases in an individual in need thereof, wherein the method comprises enhancing activity of brown adipose tissue cells in said individual by administration of a therapeutically effective amount of an agonist of Tacr2, as defined in a section “Agonist of Tacr2”, to said individual.

In further embodiments, the present disclosure relates to a method for inducing weight loss, wherein the method comprises administering a therapeutically effective amount of an agonist of Tacr2, as defined in a section “Agonist of Tacr2”, to said individual.

In other embodiments, the present disclosure relates to a method for inducing weight loss in an individual in need thereof, wherein the method comprises enhancing activity of brown adipose tissue cells in said individual by administration of a therapeutically effective amount of an agonist of Tacr2, as defined in a section “Agonist of Tacr2”, to said individual.

In some embodiments, obesity and/or obesity-associated disorders are treated by reducing the weight of the individual in need thereof of at least 5%, such as at least 6%, such as at least 7%, such as at least 8%, such as 10% or more.

Therefore, in some embodiments of the present disclosure an agonist of Tacr2 or a pharmaceutically acceptable salt thereof is administered to an individual suffering from or suspected of suffering from obesity and/or an obesity-associated diseases and causes a reduction of body weight of said individual of at least 5%, such as at least 6%, such as at least 7%, such as at least 8%, such as 10% or more.

In one embodiment, the individual in need of the treatment is an individual suffering from overweight, obesity and/or obesity-associated diseases, for example diabetes or heart diseases.

In one embodiment, the individual in need of the treatment has BMI of 25 or more, such as 30 or more, for example 35 or more, such as 40 or more. In another embodiment, the individual in need of the treatment has a waist/hip ratio of at least 0.80, for example 0.80-0.84, such as at least 0.85 (female) or at least 0.90, for example 0.9-0.99, such as above 1.00 (male). In a further embodiment, the individual in need of the treatment has fasting blood glucose of at least 6.1 mmol/l, for example at least 7.0 mmol/l. In an even further embodiment, the individual in need of the treatment has a glycated haemoglobin level of at least 42 mmol/mol, such as between 42 and 46 mmol/mol, such as at least 48 mmol/mol.

The individual in need of the treatment provided by the disclosed methods may present one or more of the following symptoms:
Elevated blood pressure: ≥140/90 mmHg;
Dyslipidemia: triglycerides (TG): ≥1.695 mmol/L and high-density lipoprotein cholesterol (HDL-C)≤0.9 mmol/L (male), ≤1.0 mmol/L (female);
Central obesity: waist:hip ratio>0.90 (male); >0.85 (female), or body mass index>30 kg/m2;
Microalbuminuria: urinary albumin excretion ratio≥20 μg/min or albumin:creatinine ratio≥30 mg/g;
Hyperglycemia: blood sugar levels higher than 11.1 mmol/l (200 mg/dl) or chronic sugar levels of 5.6-7 mmol/l (100-126 mg/dl).

Thus, the obesity-associated diseases may be a disease associated with aforementioned symptoms.

Sequences

| SEQ ID NO | SEQUENCE | NAME/NOTES |
|---|---|---|
| SEQ ID NO: 1 | HKTDSFVGLM | Neurokinin A |
| SEQ ID NO: 2 | DSFVGLM | Neurokinin A (4-10) |
| SEQ ID NO: 3 | KDSFVGXM | Neurokinin A (3-10) |
| | | [Lys3, Gly8-(R)-γ- |
| | | Lactam-Leu9]/ |
| | | GR64349 |
| | | X = NH-(R)-γ- |
| | | Lactam-Leu |
| SEQ ID NO: 4 | DSFVXLM | X = ß-Ala; Neurokinin |
| | | A (4-10) [beta-Ala8] |
| SEQ ID NO: 5 | DSFVGLX | Neurokinin A (4-10) |
| | | [Nle10]; X = Nle |
| SEQ ID NO: 6 | DSFWXLM | Neurokinin A (4-10) |
| | | [Trp7, beta-Ala8]; |
| | | X = ß-Ala |
| SEQ ID NO: 7 | YHKTDSFVGLM | Neurokinin A [Tyr0]/ |
| | | Neuromedin L [Tyr0]/ |
| | | Substance K [Tyr0] |
| SEQ ID NO: 8 | KDSXVGJM | Neurokinin A (3-10) |
| | | [Lys3, Ser5-(R)-γ- |
| | | Lactam-Leu9]; X = NH- |
| | | (R)-Phe and J = γ- |
| | | Lactam-Leu |
| SEQ ID NO: 9 | DKFVGXJ | [Lys5,Me- |
| | | Leu9, Nle10]NKA-(4- |
| | | 10); X = Me-Leu; J = Nle |
| SEQ ID NO: 10 | MGTCDIVTEANISSGPESNTTGITAFSMPSWQL | TACR2, Homo sapiens |
| | ALWATAYLALVLVAVTGNAIVIWIILAHRRMRTVT | |
| | NYFIVNLALADLCMAAFNAAFNFVYASHNIWYF | |
| | GRAFCYFQNLFPITAMFVSIYSMTAIAADRYMAI | |
| | VHPFQPRLSAPSTKAVIAGIWLVALALASPQCFY | |
| | STVTMDQGATKCVVAWPEDSGGKTLLLYHLVVI | |
| | ALIYFLPLAVMFVAYSVIGLTLWRRAVPGHQAH | |
| | GANLRHLQAMKKFVKTMVLVVLTFAICWLPYHL | |
| | YFILGSFQEDIYCHKFIQQVYLALFWLAMSSTMY | |
| | NPIIYCCLNHRFRSGFRLAFRCCPWVTPTKEDK | |
| | LELTPTTSLSTRVNRCHTKETLFMAGDTAPSEA | |
| | TSGEAGRPQDGSGLWFGYGLLAPTKTHVEI | |
| SEQ ID NO: 11 | MGAHASVTDTNILSGLESNATGVTAFSMPGWQ | TACR2, Mus musculus |
| | LALWATAYLALVLVAVTGNATVIWIILAHERMRT | |
| | VTNYFIINLALADLCMAAFNATFNFIYASHNIWYF | |
| | GSTFCYFQNLFPVTAMFVSIYSMTAIAADRYMAI | |
| | VHPFQPRLSAPSTKAVIAVIWLVALALASPQCFY | |
| | STITVDQGATKCVVAWPNDNGGKMLLLYHLVVF | |
| | VLIYFLPLVVMFAAYSVIGLTLWKRAVPRHQAHG | |
| | ANLRHLQAKKKFVKAMVLVVVTFAICWLPYHLY | |
| | FILGTFQEDIYYRKFIQQVYLALFWLAMSSTMYN | |
| | PIIYCCLNHRFRSGFRLAFRCCPWGTPTEEDRL | |
| | ELTHTPSISRRVNRCHTKETLFMTGDMTHSEAT | |
| | NGQVGGPQDGEPAGP | |
| SEQ ID NO: 12 | MKILVALAVFFLVSTQLFAEEIGANDDLNYWSD | TAC1, Homo sapiens |
| | WYDSDQIKEELPEPFEHLLQRIARRPKPQQFFG | NM_003182.2 → |
| | LMGKRDADSSIEKQVALLKALYGHGQISHKRHK | NP_003173.1 |
| | TDSFVGLMGKRALNSVAYERSAMQNYERRR | protachykinin-1 |
| | | isoform beta |
| | | precursor (129 aa) |
| SEQ ID NO: 13 | MKILVALAVFFLVSTQLFAEEIGANDDLNYWSD | TAC1, Homo sapiens |
| | WYDSDQIKEELPEPFEHLLQRIARRPKPQQFFG | NM_013997.2 → |
| | LMGKRDAGHGQISHKRHKTDSFVGLMGKRALN | NP_054703.1 |
| | SVAYERSAMQNYERRR | protachykinin-1 |
| | | isoform gamma |
| | | precursor (114 aa) |
| SEQ ID NO: 14 | MKILVAVAVFFLVSTQLFAEEIDANDDLNYWSD | TAC1, Mus musculus |
| | WSDSDQIKEAMPEPFEHLLQRIARRPKPQQFFG | NM_001311060.1 → |
| | LMGKRDAGHGQISHKRHKTDSFVGLMGKRALN | NP_001297989.1 |
| | SVAYERSAMQNYERRRK | protachykinin-1 |
| | | isoform 2 precursor |
| | | (115 aa) |
| SEQ ID NO: 15 | MKILVAVAVFFLVSTQLFAEEIDANDDLNYWSD | TAC1, Mus musculus |
| | WSDSDQIKEAMPEPFEHLLQRIARRPKPQQFFG | NM_009311.2 → |
| | LMGKRDADSSVEKQVALLKALYGHGQISHKRH | NP_033337.1 |
| | KTDSFVGLMGKRALNSVAYERSAMQNYERRRK | protachykinin-1 |
| | | isoform 1 precursor |
| | | (130 aa) |
| SEQ ID NO: 16 | DX₁FX₂X₃X₄X₅ | X₁ = S, K; X₂ = V, W; |
| | | X₃ = G, ß-Ala; |
| | | X₄ = Me-Leu, L, γ- |
| | | Lactam-Leu; X₅ = Nle, M |
| SEQ ID NO: 17 | TCATCCAGCAGGTGTTTGACA | 36B4 Fwd |
| SEQ ID NO: 18 | GGCACCGAGGCAACAGTT | 36B4 Rev |
| SEQ ID NO: 19 | GCCTCCCCACAATGTTTCTA | Tacr2 Fwd |
| SEQ ID NO: 20 | TGAGGACAAACACCACCAGA | Tacr2 Rev |
| SEQ ID NO: 21 | SKTDSFVGLM | [Ser1-Man]NKA |
| SEQ ID NO: 22 | HKTDSFVGLX | X = norleucine; |
| | | [Nle 10]NKA |
| SEQ ID NO: 23 | HKTESFVGLM | [Glu4]NKA |
| SEQ ID NO: 24 | HKTESFVGLX | X = norleucine, [Glu4, |
| | | Nle10]NKA |
| SEQ ID NO: 25 | KTDSFVGLM | NKA(2-10) |
| SEQ ID NO: 26 | KDSFVGLM | [Lys3]NKA(3-10) |
| SEQ ID NO: 27 | KESFVGLM | [Lys3, Glu4]NKA(3-10) |
| SEQ ID NO: 28 | RPKPQQFFGLM | SP |
| SEQ ID NO: 29 | DMHDFFVGLM | NKB |
| SEQ ID NO: 30 | X₆X₁FX₂X₃X₄X₅ | X6 = aspartic acid (E) |
| | | or glutamic acid (D); |
| | | X1 = serine (S) or |
| | | lysine (K); |
| | | X2 = valine (V) or |
| | | tryptophan (W); |
| | | X3 = glycine (G), ß- |
| | | alanine (ß-ala); |
| | | X4 = methyl-leucine |
| | | (Me-Leu), L, γ- |
| | | lactam-leucine (L, γ- |
| | | lactam-Leu); |
| | | X5 = norleucine (Nle), |
| | | methionine (M). |

### EXAMPLES

### Example 1 Tacr2 Expression in Adipose Tissue in Response to Cold Exposure and High Fat Diet

### Animals

All animal experiments were performed according to Danish animal legislation, with ad libitum access to food and water and 12 hour light-dark cycle. Male C57Bl/6 mice were purchased from Taconic (Denmark) at four weeks of age and acclimatized for one week prior to experimentation. For cold-exposure studies, mice were randomized according to their weight and body composition measured by MRI scan (EchoMRI, TX, USA) before single-housing at thermoneutrality (app 28° C.) for at least three weeks prior to cold-exposure. For high fat diet studies, male C57Bl/6 mice were housed at room temperature and fed a 60% high fat diet (Research diets, D12492, Denmark) for 7 weeks.

### Adipocyte Fractionation

Intrascapular BAT from four weeks old male C57Bl/6 mice housed at thermoneutral or cold was digested with collagenase Type II (Sigma-Aldrich, Denmark) on horizontal shaking water bath at 37° C. for 30 minutes with vortexing every 10 minutes. Stromel vascular fraction (SVF) and adipocyte fractions were separated by centrifugation.

### RNA Extraction

Tissue was lysed using Tri-Reagent (Invitrogen, Denmark) and homogenized using Tissue Lyser (Thermo, Denmark). RNA was extracted with Qiagen RNeasy Mini Kit (Germany) following manufacturer's protocol.

qPCR

Complementary DNA was made using AB High Capacity cDNA Reverse Transcription Kit (Applied Biosystems, Denmark) following manufacturer's protocol, and qPCR was performed using Sybr Green (Primer Design, UK) on Roche LC480 (Roche, Switzerland). Primers were purchased from Tag Copenhagen (Denmark), see Table 2:

TABLE 2
Primers used for qPCR

| Primer | Sequence |
|---|---|
| 36B4 Fwd | Tcatccagcaggtgtttgaca |
| 36B4 Rev | Ggcaccgaggcaacagtt |
| Tacr2 Fwd | Gcctccccacaatgtttcta |
| Tacr2 Rev | Tgaggacaaacaccaccaga |

### Results

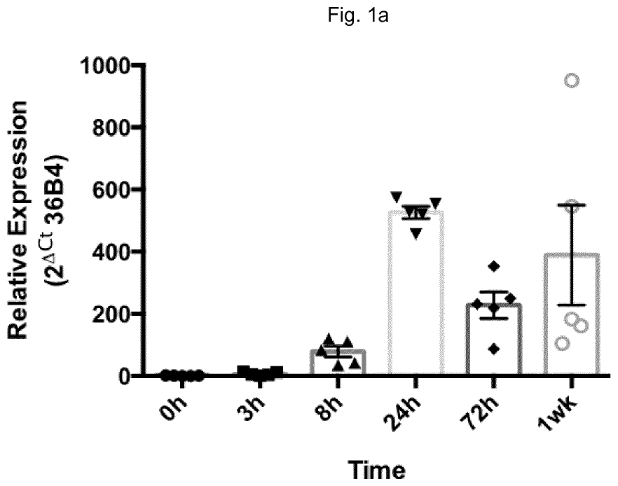

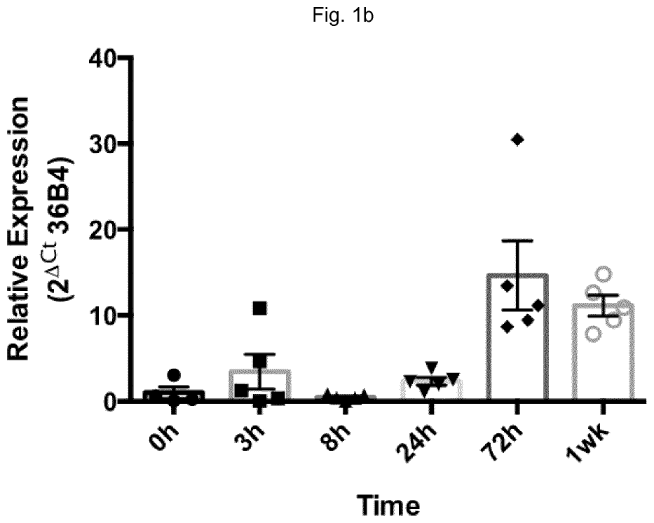

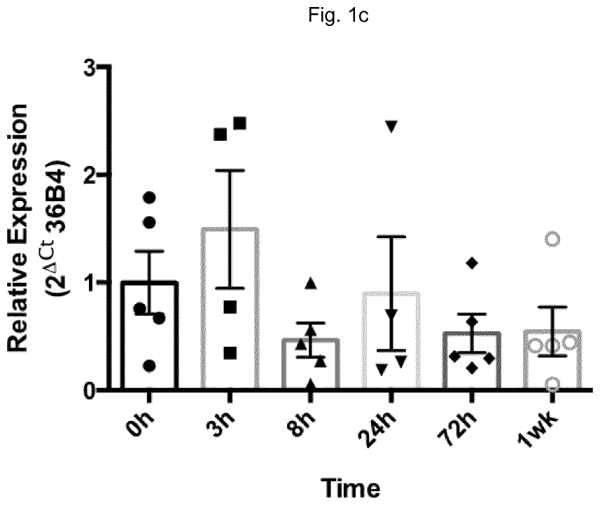

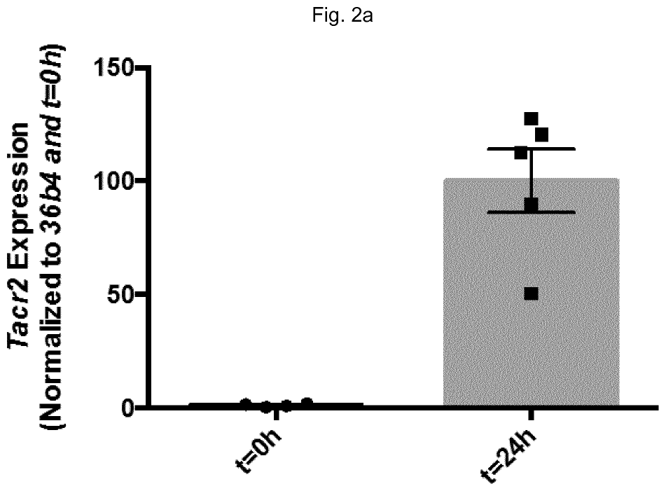

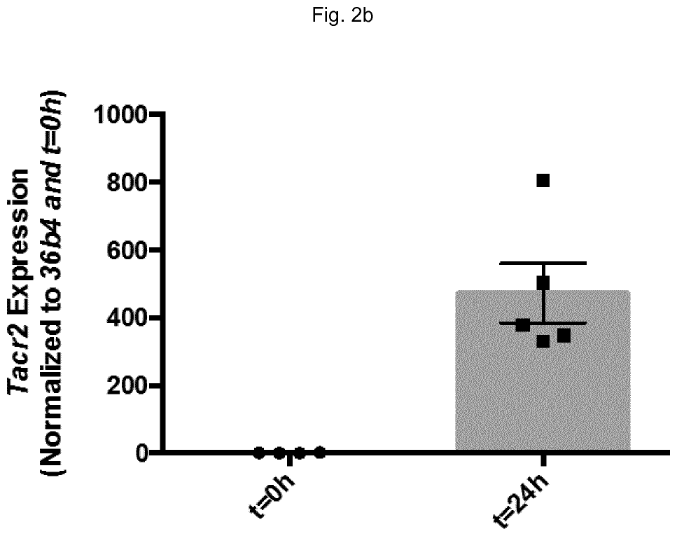

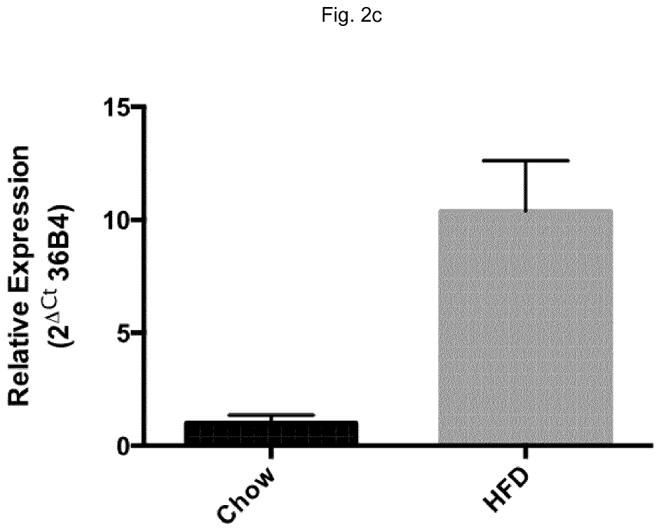

The in vivo experiments indicate that Tacr2 is significantly induced in interscapular brown adipose tissue (iBAT) and inguinal white adipose tissue (iWAT) following cold-exposure (4° C.) compared to thermoneutrality (30° C.). Notably, cold-induced expression of Tacr2 in iBAT and iWAT remains markedly induced at the end of a 1-week cold exposure whereas epididymal white adipose tissue (eWAT), which does not undergo browning or elicit appreciable energy expenditure, does not induce Tacr2 expression upon cold challenge (FIG. 1a-c). The cold mediated induction of Tacr2 in BAT is occurring in both mature adipocytes and pre-adipocyte stem cells (FIG. 2a-b). Furthermore, 60% high fat diet challenge increases Tacr2 expression levels in brown adipocytes suggesting a therapeutic sensitization to administration of Tacr2 agonists, such as NKA in obese individuals (FIG. 2c).

Results: Expression of Tacr2 in brown adipocytes, pre-adipocytes stem cells and iWAT was induced in response to exposure to cold. Tacr2 expression levels increase in brown adipocytes also in response to high fat diet.

### Example 2: Inositol 3-phosphate (IP3) Levels and O₂ Consumption in Primary Brown Adipocytes Treated With NKA

### Primary Adipocyte Isolation, Differentiation and Culture

Primary brown pre-adipocytes were isolated from intrascapular brown fat pads by collagenase type 1 (Worthington, NJ, USA) digestion on horizontal shaking water bath at 37° C. for 50 minutes with vortexing every 10 minutes. Collagenase was diluted in KRB with free fatty acid free BSA (Sigma-Aldrich, Denmark) in a ratio of 1:10. The enzymatic reaction was stopped by addition of growth media, and cell suspension was pelleted, resuspended in growth media before straining through a 40 μm nylon mesh (VWR, Denmark) to avoid tissue clumps. Pre-adipocytes were expanded in DMEM (Gibco, Denmark) supplemented with 10% fetal bovine serum (Invitrogen, Denmark) and 1% penicillin and streptomycin (Lonza, Denmark) until confluency. The day after reach of confluency, primary brown pre-adipocytes were differentiated using a cocktail consisting of insulin (0.5 mg/mL), rosiglitazone (1 mM), dexamethasone (100 μM), IBMX (50 mM) and T3 (1 μM) for two days, thereafter cells were maintained in growth media containing insulin and T3 throughout the experimental period. Maintenance media was changed every second day. Cells were grown at 37° C. with 10% CO₂.

### Transient Transfection and Treatment

At day three of differentiation, cells were transiently transfected by electroporation or replated for experiments. For transient transfection, pcDNA3.1 (Genscript, NJ, USA) containing mouse Tacr2 coding sequence or empty were electroporated into the adipocytes using electroporation machine program A34 and kit from Lonza (Denmark). Adipocytes were treated with Neurokinin A (10-7M) for indicated time.

### IP3 Assay

Primary brown adipocytes were loaded with 5 μCi myo-[3H]inositol (Perkin Elmer, Denmark) in full maintenance media at the day after electroporation, and incubated overnight. Cells were treated with NKA for 90 minutes at 37° C. in HBSS supplemented with 10 mM LiCl. After treatment, cells were extracted with 10 mM formic acid and cell membranes were captured using poly-L-lysine coated SPA beads (Perkin Elmer, Denmark), and analyzed on Top Counter (Perkin Elmer, Denmark).

### Oxygen Consumption

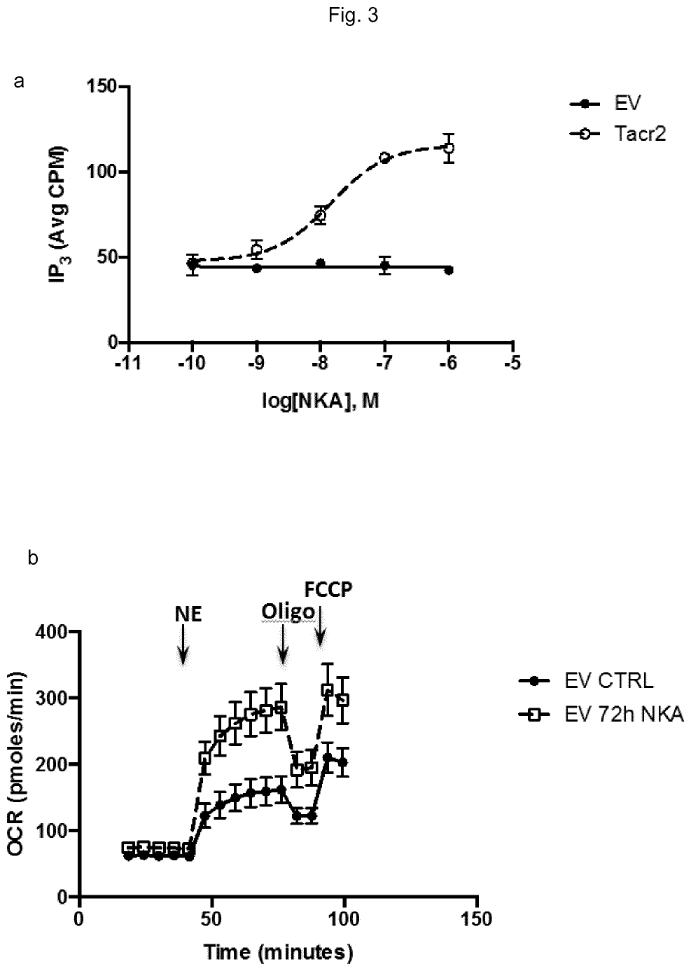

Oxygen consumption was measured using the Seahorse XF-96 Flux Analyzer (Seahorse Bioscience, Denmark). Primary brown adipocytes were seeded in Seahorse 96-well cell culture plates (Seahorse Biosciences, Denmark) in full maintenance media at the day after electroporation, and cultured for an additional four days before experimentation. Cells were allowed to equilibrate in Flux media consisting of unbuffered DMEM without phenol red (Sigma-Aldrich, Denmark) supplemented with 0.5% free fatty acid free BSA (Sigma-Aldrich, Denmark), 25 mM glucose and 1 mM pyruvate (Company, Country), and pH was adjusted to 7.4. All readings were performed over a two minutes period and mixing was performed for three minutes. Neurokinin A (NKA) of SEQ ID NO:1 containing a C-terminal amidation was added to the cells for 24 h before IP3 determination and for 72 h before start of determining Oxygen consumption (time 0 of FIG. 3b). Compounds were diluted in Flux media and injected to the cell culture at the times indicated in FIG. 3b to a final concentration of: norepinephrine (5 μM), oligomycin (1 μM), FCCP (1 μM), antimycin A (1 μM) and rotenone (1 μM). The experiments were conducted on primary adipocytes exposed to NKA for 72 hours (FIG. 3b)

### Results:

The in vitro experiments indicate that 24 hours NKA treatment increases IP3 levels in Tacr2 over-expressing primary brown adipocytes (FIG. 3a). The experiments also show that 72 hours NKA treatment increases oxygen consumption rate (OCR) also in primary brown adipocytes not transfected with Tacr2 (FIG. 3b).

The increased oxygen consumption indicates an increased energy expenditure of the brown adipocytes in response to NKA.

### Example 3

Glucose uptake studies are performed on primary brown and beige/brite adipocytes in Tacr2 loss-of-function studies, using conditional and whole body knockout mice, following administration with either Neurokinin A (NKA) of SEQ ID NO:1 containing a C-terminal amidation or vehicle. Additionally, NKA mediated agonism on respiratory capacity of human brown and beige/brite adipocytes is tested. The energy expenditure is assessed via PET scans.

### Example 4: Effect of TACR2 Agonism on Thermogenesis and on Reversion of Obesity and Diabetes-Related Complications

### High Fat Diet (HFD)

Five week old male C57Bl/6 mice are fed a HFD with 60% energy from fat until obese (50 g) and glucose intolerant as model of type 2 diabetes (T2D).

### Leptin Deficient (Ob/Ob) Mice

Ob/Ob mice are fed a chow diet until obese and glucose intolerant as genetic model of T2 D.

### Multiple Low-Dose Streptozotocin (MLDS)

Seven weeks old male C57Bl/6 mice fed chow diet are administered with daily injections of streptozotocin (35 mg per kg) for five consecutive days (Hansen J B et al. 2012) as a model of type 1 diabetes (T1D).

### Insulin Tolerance Test (ITT)

Obese mice are injected with insulin i.p., and glucose levels are assessed over two hours.

### Glucose Tolerance Test (GTT)

Obese mice are injected with glucose i.p. or given an oral gavage with glucose, and glucose levels are assessed over two hours.

### Weight and Body Composition

Weight and body composition (by MRI scan) is monitored throughout the experiment.

### Energy Expenditure

Throughout treatment with NKA/Vehicle, mice are housed in the TSE indirect calorimetry system, to measure food and water intake, physical activity, 02 consumption and CO₂ production from the mice.

### Example 5

### Animals

All animal experiments were performed according to Danish animal legislation, with ad libitum access to food and water and 12 hour light-dark cycle. Male C57Bl/6 mice were purchased from Janvier (Denmark) at four weeks of age and acclimatized for one week prior to experimentation. For high fat diet studies, male C57Bl/6 mice were housed at room temperature and fed a 60% high fat diet (Research diets, D12492, Denmark) for 18 weeks. After being fed this diet, the mice were highly obese, typically having a body weight in the range of 42 to 45 g. These mice may also be referred to as diet induced obese (DIO) mice. Mice were randomized according to their weight and body composition measured by MRI scan (EchoMRI, TX, USA) before single-housing for at least one week prior treatment. Oxygen consumption and respiratory efficiency rate (RER) were measured using PhenoMaster (TSE-Systems, Germany). Food consumption was manually assessed every second day.

### Neurokinin A Treatment

Neurokinin A (NKA) of SEQ ID NO:1 was custom synthesized by Almac Group (UK) and dissolved in sterile saline solution. For treatment, NKA was diluted in Gelofusine® (B. Braun, DK) prior to subcutaneous injection. Mice were treated twice daily for 9 days.

### Insulin Tolerance Test

Mice were fasted for 3 hours prior to intra peritoneal insulin tolerance test (IPITT). Insulin (Actrapid, Novo Nordisk, DK) was dosed at 1 IU per kg lean body mass measured prior to treatment. Blood glucose was determined using glucometer from Roche. Lower blood glucose levels indicate greater insulin sensitivity.

### Results

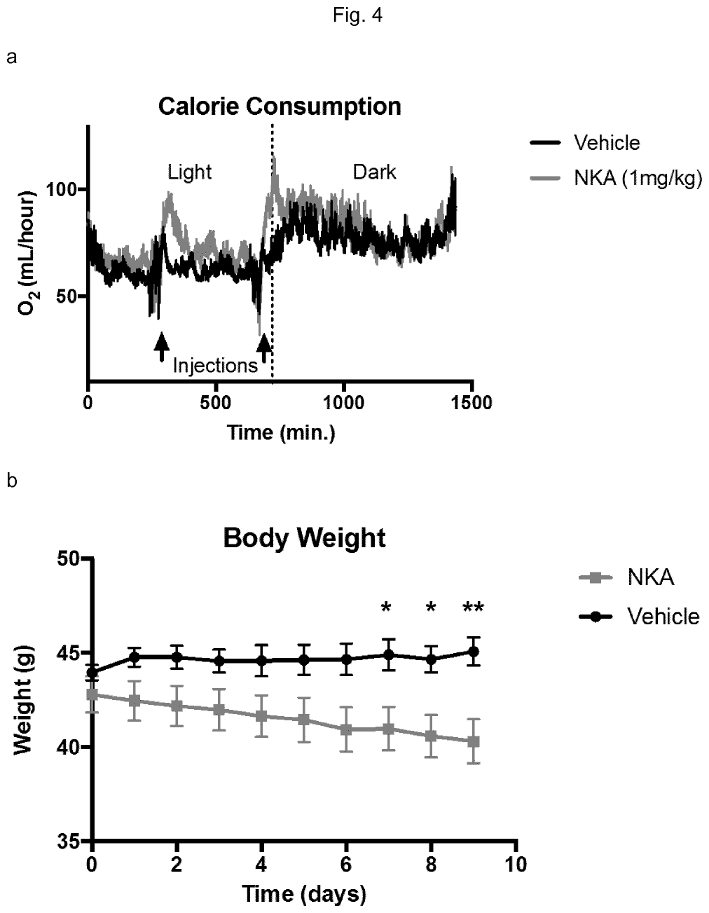

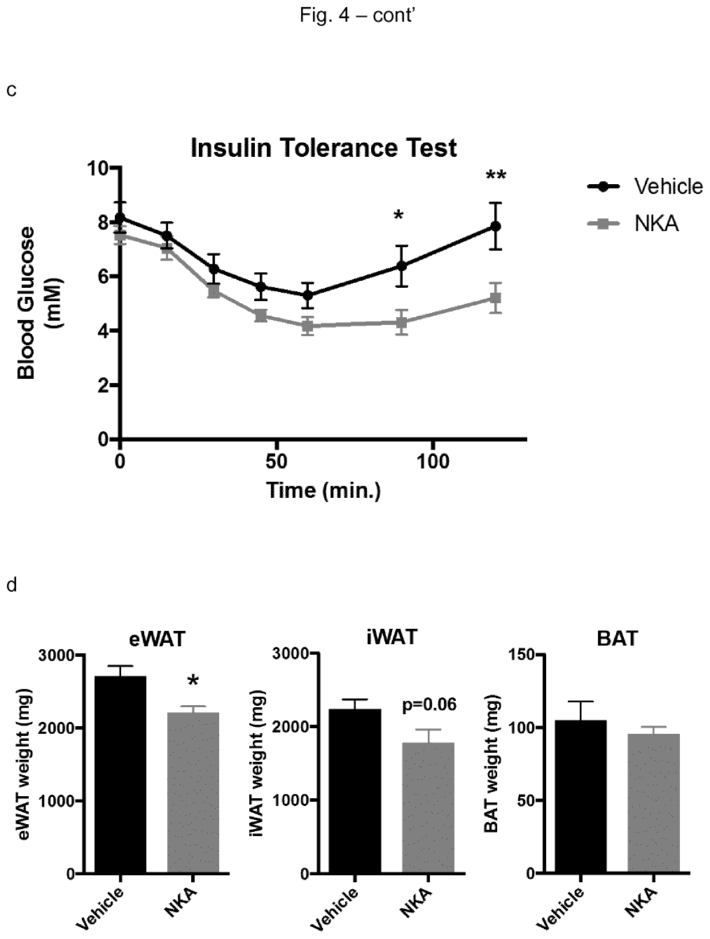

Neurokinin A treatment increased calorie burning (FIG. 4a) and improved metabolic parameters in diet induced obese (DIO) mice. Interestingly, the calorie burning was not accompanied by desensitization to NKA, in fact the results shown in FIG. 4a are representative for the 9 consecutive days of treatment. A significant reduction in body weight was also observed (FIG. 4b), which was due to a reduction of white adipose tissue and not brown fat as shown in FIG. 4d. In addition, the ITT showed that the treated mice had a significant lower blood glucose level after insulin treatment and thus a higher insulin sensitivity than the control mice. Interestingly, the blood glucose levels not only are lower but, after having reached Cₘᵢₙ, also return to basal levels at a slower rate.

### Example 6

### Peptide Design and Synthesis

NKA was designed to bind albumin via fatty acid acylation at the N-terminal or to gamma-Glu attached on Lys2. Peptides were labeled with myristic acid (C14), palmitic acid (C16) or stearic acid (C18). NKA was additionally glycosylated using mannose coupled to a Ser residue.

Peptides were synthesized by Almac Group (Scotland), using resin synthesis and purified by RP-HPLC. Peptides were analyzed by HPLC and mass spectrometry.

### Example 7

### IP3 Assay

HEK293 cells were transiently transfected with plasmid (pcDNA3.1, Genscript, NJ, USA) encoding human or mouse Tacr1-3 using Lipofectamine 2000 (Thermo Fisher, Denmark). After six hours of transfection, cells were loaded with 5 μCi myo-[3H]inositol (Perkin Elmer, Denmark) in full maintenance and cultured overnight. Cells were treated with NKA analogues for 90 minutes at 37° C. in HBSS supplemented with 10 mM LiCl. After treatment, cells were extracted with 10 mM formic acid and cell membranes were captured using poly-L-lysine coated SPA beads (Perkin Elmer, Denmark), and analyzed on Top Counter (Perkin Elmer, Denmark).

NKA analogues were dissolved in DMSO and diluted in PBS containing 0.2% or 2% w/v free fatty acid (FFA)-free bovine serum albumin (BSA) (Sigma, Denmark).

Data was analyzed using GraphPad Prism 7 (GraphPad, CA, USA),using 4 parametric logistic curve fitting. The data analysis included calculation of the top value, the bottom value, the hill slope as well as the log EC50.

### Weight Loss

Diet induced obese (DIO) mice were prepared as described in Example 5 above. DIO mice were treated with once daily with 1 mg/kg [Lys2-γ-Glu-C16]NKA or vehicle (sterile filtered saline solution containing 3% w/v FFA-free BSA and 1.25% DMSO) s.c. for three consecutive days followed by a washout period and an additional four treatments. The mice were monitored after treatment to observe lasting effects of the treatment. Food intake and calorie-burning was monitored using PhenoMaster (TSE-Systems, Germany).

Results: The results of the IP3 assay show that NKA and NKA analogues (SEQ ID NO:1 and 21, labeled with myristic acid (C14), palmitic acid (C16), stearic acid (C18), or mannose) are specific for Tacr2 receptor, both in the case of murine and human receptors; see table 3, 4 and 5.

TABLE 3
Tacr1 activation data of NKA, SP, NKA analogues (SEQ ID NO:
1 and 21, labeled with myristic acid (C14), palmitic acid
(C16), stearic acid (C18), or mannose) and stable NKA analogues
(SEQ ID NO: 2, 5 and 22 to 27) relative to Tacr1.

| | Top | Bottom | LogEC50 | HillSlope |
|---|---|---|---|---|
| | mTacr1 | | | |
| NKA | 1099 | 186.2 | −7.859 | 1.224 |
| [Nle10]NKA | 822.9 | 120.8 | −6.914 | 1.017 |
| [Glu4]NKA | 976.4 | 150.5 | −7.496 | 0.8386 |
| [Glu4, Nle10]NKA | 762.2 | 155.1 | −6.955 | 1.697 |
| NKA(2-10) | 1794 | 111.4 | −6.672 | 0.4862 |
| [Lys3]NKA(3-10) | 1058 | 79.14 | −7.089 | 0.5992 |
| [Lys3, Glu4]NKA(3-10) | 992.3 | 103 | −7.091 | 0.8347 |
| NKA(4-10) | 1030 | 99.29 | −7.177 | 0.6407 |
| [Nle10]NKA(4-10) | 717.5 | 214.8 | ~−6.868 | ~5.408 |
| [Lys2-γ-Glu-C14]NKA | 1187 | 158.6 | −7.774 | 0.8251 |
| [Lys2-γ-Glu-C18]NKA | 1287 | 53.31 | −8.253 | 0.3855 |
| [Ser1-Man]NKA | 945.9 | 171.8 | −7.877 | 0.8673 |
| [Lys2-γ-Glu-C16]NKA | 1701 | 76.,86 | −7.153 | 0.4163 |
| C16-NKA | 950.1 | 138.4 | −7.182 | 1.077 |
| SP | 1226 | 170.6 | −8.119 | 1.317 |
| | hTACR1 | | | |
| NKA | 2939 | 342.7 | −8.111 | 1.216 |
| [Nle10]NKA | 2783 | 320.1 | −7.014 | 0.9527 |
| [Glu4]NKA | 2984 | 300.7 | −7.667 | 0.8248 |
| [Glu4, Nle10]NKA | 2255 | 293.2 | −7.05 | 1.106 |
| NKA(2-10) | 3037 | 294.5 | −7.93 | 0.8844 |
| [Lys3]NKA(3-10) | 2837 | 305.3 | −7.717 | 1.07 |
| [Lys3, Glu4]NKA(3-10) | 2871 | 276.6 | −7.381 | 0.997 |
| NKA(4-10) | 2825 | 248.1 | −7.765 | 1.115 |
| [Nle10]NKA(4-10) | 2510 | 281.2 | −6.909 | 1.153 |
| [Lys2-γ-Glu-C14]NKA | 3025 | 293.2 | −7.84 | 1.068 |
| [Lys2-γ-Glu-C18]NKA | 2797 | 315.9 | −8.756 | 2.037 |
| [Ser1-Man]NKA | 2702 | 290.1 | −8.301 | 1.111 |
| [Lys2-γ-Glu-C16]NKA | 3033 | 291.5 | −8.047 | 0.9213 |
| C16-NKA | 2566 | 269 | −7.034 | 1.324 |
| SP | 3242 | 312 | −7.98 | 1.297 |

TABLE 4
Tacr2 activation data of NKA, NKA analogues (SEQ ID NO: 1
and 21, labeled with myristic acid (C14), palmitic acid
(C16), stearic acid (C18), or mannose) and stable NKA analogues
(SEQ ID NO: 2, 5 and 22 to 27) relative to Tacr2.

| | Top | Bottom | LogEC50 | HillSlope |
|---|---|---|---|---|
| | mTacr2 | | | |
| NKA | 1759 | 212.4 | −8.092 | 0.9825 |
| [Nle10]NKA | 1268 | 165.9 | −7.695 | 0.8214 |
| [Glu4]NKA | 1855 | 177.2 | −7.495 | 0.7149 |
| [Glu4, Nle10]NKA | 1738 | 175.8 | −7.204 | 0.9053 |
| NKA(2-10) | 2953 | 10.31 | −7.012 | 0.3755 |
| [Lys3]NKA(3-10) | 2131 | 53.73 | −7.012 | 0.4661 |
| [Lys3, Glu4]NKA(3-10) | 2676 | 27.69 | −6.733 | 0.3747 |
| NKA(4-10) | 1920 | 120.1 | −7.665 | 0.5803 |
| [Nle10]NKA(4-10) | 1792 | 167.8 | −7.222 | 0.6174 |
| [Lys2-γ-Glu-C14]NKA | 2054 | 86.18 | −8.041 | 0.6378 |
| [Lys2-γ-Glu-C18]NKA | 1822 | 96.79 | −9.06 | 0.6845 |
| [Ser1-Man]NKA | 1908 | 153.4 | −8.202 | 0.7859 |
| [Lys2-γ-Glu-C16]NKA | 2748 | −46.72 | −7.716 | 0.3756 |
| C16-NKA | 2150 | 124.3 | −6.869 | 0.5286 |
| | hTACR2 | | | |
| NKA | 2798 | 370.6 | −8 | 0.7933 |
| [Nle10]NKA | 2242 | 336.8 | −8.032 | 0.7031 |
| [Glu4]NKA | 3194 | 210.9 | −7.53 | 0.4594 |
| [Glu4, Nle10]NKA | 2054 | 308.1 | −7.819 | 0.6965 |
| NKA(2-10) | 2753 | 295.5 | −8.173 | 0.6936 |
| [Lys3]NKA(3-10) | 2812 | 267.4 | −8.087 | 0.6458 |
| [Lys3, Glu4]NKA(3-10) | 2819 | 261.7 | −7.785 | 0.6009 |
| NKA(4-10) | 2686 | 289 | −8.014 | 0.6529 |
| [Nle10]NKA(4-10) | 2393 | 241.5 | −7.821 | 0.439 |
| [Lys2-γ-Glu-C14]NKA | 3052 | 269.5 | −7.41 | 0.4837 |
| [Lys2-γ-Glu-C18]NKA | 2337 | 321.6 | −8.585 | 0.678 |
| [Ser1-Man]NKA | 2683 | 273.1 | −8.101 | 0.6016 |
| [Lys2-γ-Glu-C16]NKA | 2989 | 297.7 | −7.973 | 0.6044 |
| C16-NKA | 2166 | 319.1 | −7.205 | 0.9049 |

TABLE 5
Tacr3 activation data of NKA, NKB, NKA analogues (SEQ ID NO:
1 and 21, labeled with myristic acid (C14), palmitic acid
(C16), stearic acid (C18), or mannose) and stable NKA analogues
(SEQ ID NO: 2, 5 and 22 to 27) relative to Tacr3.

| | Top | Bottom | LogEC50 | HillSlope |
|---|---|---|---|---|
| | mTacr3 | | | |
| NKA | 1386 | 191.2 | −7.362 | 0.9773 |
| [Nle10]NKA | 909 | 153.7 | ~−6.975 | ~5.939 |
| [Glu4]NKA | 1275 | 174.9 | −6.905 | 0.9939 |
| [Glu4, Nle10]NKA | 1097 | 191.5 | ~−6.851 | ~4.147 |
| NKA(2-10) | 2208 | 154.3 | −6.479 | 0.6882 |
| [Lys3]NKA(3-10) | 2006 | 116.3 | −6.409 | 0.8658 |
| [Lys3, Glu4]NKA(3-10) | 1651 | 138 | −6.605 | 1.25 |
| NKA(4-10) | 2065 | 137.4 | −6.836 | 0.8164 |
| [Nle10]NKA(4-10) | 1621 | 183.2 | −6.754 | 1.214 |
| [Lys2-γ-Glu-C14]NKA | 1298 | 192.9 | −8.61 | 1.326 |
| [Lys2-γ-Glu-C18]NKA | 1091 | 218.7 | −9.32 | 1.351 |
| [Ser1-Man]NKA | 1135 | 186.9 | −7.693 | 1.527 |
| [Lys2-γ-Glu-C16]NKA | 1596 | 231.6 | −9.081 | 1.874 |
| C16-NKA | 1379 | 185 | −8.516 | 1.359 |
| NKB | 1288 | 219.9 | −7.881 | 1.32 |
| | hTACR3 | | | |
| NKA | 4737 | 335.5 | −7.214 | 1.127 |
| [Nle10]NKA | 4267 | 309.7 | −6.516 | 1.201 |
| [Glu4]NKA | 4512 | 296.3 | −6.759 | 1.096 |
| [Glu4, Nle10]NKA | 2403 | 305.9 | ~−6.819 | ~4.572 |
| NKA(2-10) | 4994 | 298.9 | −6.989 | 0.8505 |
| [Lys3]NKA(3-10) | 5029 | 284.3 | −6.635 | 0.8997 |
| [Lys3, Glu4]NKA(3-10) | 4419 | 271.1 | −6.493 | 1.031 |
| NKA(4-10) | 4060 | 278.7 | −7.044 | 1.09 |
| [Nle10]NKA(4-10) | 3865 | 295.3 | −6.38 | 1.076 |
| [Lys2-γ-Glu-C14]NKA | 4924 | 307.5 | −7.963 | 1.033 |
| [Lys2-γ-Glu-C18]NKA | 4396 | 325 | −8.732 | 1.241 |
| [Ser1-Man]NKA | 3558 | 292.9 | −7.552 | 1.304 |
| [Lys2-γ-Glu-C16]NKA | 4565 | 321.2 | −8.374 | 1.253 |
| C16-NKA | 6906 | 303 | −7.08 | 0.9154 |
| NKB | 4892 | 331.9 | −7.689 | 1.111 |

The results of the IP3 assay show that stable NKA analogues (SEQ ID NO: 2, 5 and 22 to 27) are specific for Tacr2 receptor, both in the case of murine and human receptors; see table 4.

NKA and NKA analogues (SEQ ID NO:1 and 21, labeled with myristic acid (C14), palmitic acid (C16) or stearic acid (C18), have low BSA binding affinity, indicating that they are specific for Tacr2, as shown in Table 5.

TABLE 5
Tcar2 activation of BSA. BSA binding of NKA and NKA analogues (SEQ ID NO: 1 and
21, labeled with myristic acid (C14), palmitic acid (C16) or stearic acid (C18)

| | mTacr2, 0.2% BSA | | | | mTacr2, 2% BSA | | | |
|---|---|---|---|---|---|---|---|---|
| | | | | Hill- | | | | Hill- |
| | Top | Bottom | LogEC50 | Slope | Top | Bottom | LogEC50 | Slope |
| NKA | 1788 | 138.7 | −8.451 | 1.082 | 1719 | 111.3 | −7.909 | 0.8378 |
| [Lys2-γ-Glu- | 1512 | 98.92 | −7.891 | 0.988 | 1347 | 95.82 | −7.104 | 1.033 |
| C14]NKA | | | | | | | | |
| [Lys2-γ-Glu- | 1424 | 113.8 | −8.238 | 1.156 | 1154 | 44.14 | −8.084 | 0.6676 |
| C18]NKA | | | | | | | | |
| [Lys2-γ-Glu- | 1643 | 102.5 | −8.355 | 0.9326 | 962.9 | 91.27 | −8.509 | 1.603 |
| C16]NKA | | | | | | | | |
| C16-NKA | 1540 | 101 | −7.121 | 0.938 | 1268 | 101.9 | −6.283 | 0.8399 |

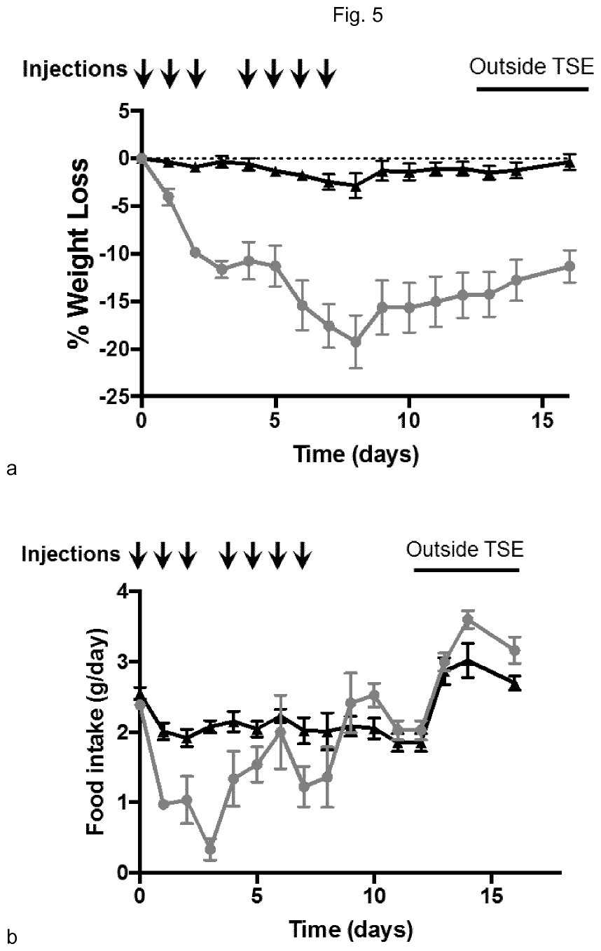

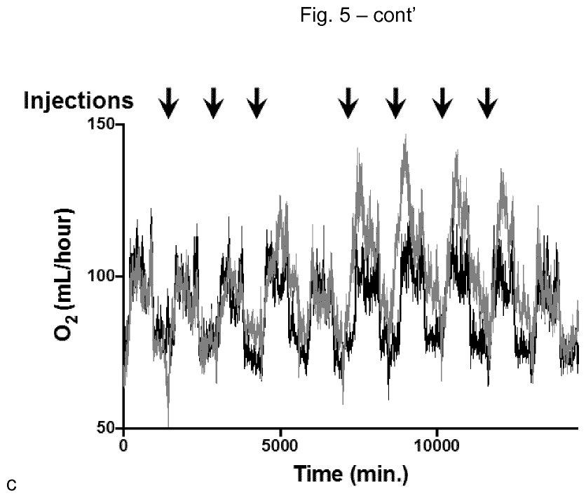

A significant reduction in body weight was observed (FIG. 5a) as result of treatment with [Lys2-γ-Glu-C16]NKA, which was due to a reduction of white adipose tissue and not brown fat. Food intake was significantly reduced when treating mice with [Lys2-γ-Glu-C16]NKA. Treatment with [Lys2-γ-Glu-C16]NKA also increased calorie burning as shown by the reduction in body weight (FIG. 5c) and improved metabolic parameters in diet induced obese (D10) mice.

### REFERENCES

Ablas J., van Etten I. Khanum A. et al., 1995. C-terminal truncation of the neurokinin-2 receptor causes enhanced and sustained agonist-induced signalling. The Journal of Biological Chemistry, 270, 8944-8951.
Bellucci F, Carini F, Catalani C, Cucchi P, Lecci A, Meini S, Patacchini R, Quartara L, Ricci R, Tramontana M, Giuliani S, Maggi C A. (2002) Pharmacological profile of the novel mammalian tachykinin, hemokinin 1. Br J Pharmacol, 135: 266-274.
Bellucci F. et al. 2002. Pharmacological profile of the novel mammalian tachykinin, hemokinin 1, British Journal of Pharmacology 135:266-274.
Cannon and Nedergaard, 2004. Brown adipose tissue: function and physiological significance. Physiol. Rev., 84(1):277-359.
Cypess A. M., Weiner L. S., Roberts-Toler C., Franquet Elía E., Kessler S. H., Kahn P. A., English J., Chatman K., Trauger S. A., Doria A., Kolodny G. M., 2015. Activation of human brown adipose tissue by a β3-adrenergic receptor agonist. Cell Metabolism, 21(1):33-8.
Deal M J, Hagan R M, Ireland S J, Jordan C C, McElroy A B, Porter B, Ross B C, Stephens-Smith M, Ward P. (1992) Conformationally constrained tachykinin analogues: potent and highly selective neurokinin NK-2 receptor agonists. J. Med. Chem., 35 (22): 4195-204.
D'Orléans-Juste P, Dion S, Drapeau G, Regoli D. (1986) Different receptors are involved in the endothelium-mediated relaxation and the smooth muscle contraction of the rabbit pulmonary artery in response to substance P and related neurokinins. Eur. J. Pharmacol., 125 (1): 37-44.
Douglas S. D., Leeman S. E., Barrett J., Dombrowsky E., Heyward C. Y., Remeshwar P. Tachykinin receptors: NK2 receptor. Last modified on 02/04/2015. Accessed on 17 Mar. 2016. IUPHAR/BPS Guide to pharmacology, http://wwvv.guidetopharmacolociy.org/GRAC/ObjectDisplayForward?objectId=361.
Emonds-Alt X, Golliot F, Pointeau P, Le Fur G, Breliere J C. (1993) Characterization of the binding sites of [3H]SR 48968, a potent nonpeptide radioligand antagonist of the neurokinin-2 receptor. Biochem Biophys Res Commun, 191: 1172-1177.
Hansen J B, Tonnesen M F, Madsen A N, Hagedorn P H, Friberg J, Grunnet L G, Heller R S, Nielsen A Ø, Størling J, Baeyens L, Anker-Kitai L, Qvortrup K, Bouwens L, Efrat S, Aalund M, Andrews N C, Billestrup N, Karlsen A E, Hoist B, Pociot F, Mandrup-Poulsen T. (2012). Divalent metal transporter 1 regulates iron-mediated ROS and pancreatic β cell fate in response to cytokines. Cell Metab. 16(4):449-61.
Matuszek M A, Zeng X P, Strigas J, Burcher E. (1998) An investigation of tachykinin NK2 receptor subtypes in the rat. Eur. J. Pharmacol., 352 (1): 103-9.
Remington: The Science and Practice of Pharmacy 2005, Lippincott, Williams & Wilkins.
Sarau H M, Griswold D E, Potts W, Foley J J, Schmidt D B, Webb E F, Martin L D, Brawner M E, Elshourbagy N A, Medhurst A D, Giardina G A, Hay D W. (1997) Nonpeptide tachykinin receptor antagonists: I. Pharmacological and pharmacokinetic characterization of SB 223412, a novel, potent and selective neurokinin-3 receptor antagonist. J Pharmacol Exp Ther, 281: 1303-1311.
van Giersbergen P L, Shatzer S A, Burcher E, Buck S H. (1992) Comparison of the effects of neuropeptide K and neuropeptide gamma with neurokinin A at NK2 receptors in the hamster urinary bladder. Naunyn Schmiedebergs Arch. Pharmacol., 345 (1): 51-6.
Warner F J, Comis A, Miller R C, Burcher E. (1999) Characterization of the [125l]-neurokinin A binding site in the circular muscle of human colon. Br. J. Pharmacol., 127 (5): 1105-10.
Warner F J, Mack P, Comis A, Miller R C, Burcher E. (2001) Structure-activity relationships of neurokinin A (4-10) at the human tachykinin NK(2) receptor: the role of natural residues and their chirality. Biochem Pharmacol, 61: 55-60.

1. An agonist of Tacr2 or a pharmaceutically acceptable salt thereof for use in treatment of insulin resistance, diabetes and/or obesity in an individual in need thereof.

2. An agonist of Tacr2 or a pharmaceutically acceptable salt thereof for use in a method of inducing weight loss in an individual in need thereof.

3. The agonist according to any one of the preceding claims, wherein the agonist comprises or consists of a peptide.

4. The agonist according to any one of the preceding claims, wherein the agonist consists of a peptide optionally linked to a conjugated moiety.

5. The agonist of any one of the preceding claims, wherein the agonist or the peptide comprises or consist of a peptide of sequence X₆X₁FX₂X₃X₄X₅and wherein
X₆=aspartic acid (E) or glutamic acid (D);
X₁=serine (S) or lysine (K);
X₂=valine (V) or tryptophan (W);
X₃=glycine (G), β-alanine (β-ala);
X₄=methyl-leucine (Me-Leu), L, γ-lactam-leucine (γ-lactam-Leu);
X₅=norleucine (Nle), methionine (M). 25 6. The agonist of any one of the preceding claims, wherein the agonist or the peptide comprises or consist of a peptide of sequence DX₁FX₂X₃X₄X₅and wherein
X₁=serine (S) or lysine (K);
X₂=valine (V) or tryptophan (W);
X₃=glycine (G), β-alanine (β-ala);
X₄=methyl-leucine (Me-Leu), L, γ-lactam-leucine (γ-lactam-Leu);
X₅=norleucine (Nle), methionine (M).

7. The agonist of any one of the preceding claims, wherein the agonist or the peptide comprises or consists of Neurokinin A or a functional analogue thereof.

8. The agonist of any one of claims 4 to 7, wherein the peptide consists of at least 7 and at most 130 amino acid residues.

9. The agonist of any one of claims 4 to 7, wherein the peptide consists of at least 10 and at most 130 amino acid residues.

10. The agonist of any one of claims 4 to 9, wherein the peptide comprises a precursor protein of Neurokinin A of SEQ ID NO:12, SEQ ID NO:13, SEQ ID NO:14 or SEQ ID NO:15 or a functional analogue thereof sharing at least 75% sequence identity.

11. The agonist of any one of claims 4 to 10, wherein the peptide comprises a fragment of the precursor protein of Neurokinin A of SEQ ID NO:12, SEQ ID NO:13, SEQ ID NO:14 or SEQ ID NO:15 or a functional analogue thereof sharing at least 75% sequence identity.

12. The agonist of any one of claims 4 to 11, wherein the peptide consists of a consecutive sequence of SEQ ID NO:12, SEQ ID NO:13, SEQ ID NO:14 or SEQ ID NO:15 or a functional analogue thereof sharing at least 75% sequence identity.

13. The agonist of any one of claims 4 to 12, wherein the peptide comprises a sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:21, SEQ ID NO:22, SEQ ID NO:23, SEQ ID NO:24, SEQ ID NO:25, SEQ ID NO:26, SEQ ID NO:27and wherein at the most 2 amino acids have been exchanged.

14. The agonist of any one of claims 4 to 13, wherein the peptide consists of a sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:21, SEQ ID NO:22, SEQ ID NO:23, SEQ ID NO:24, SEQ ID NO:25, SEQ ID NO:26, SEQ ID NO:27and wherein at the most 2 amino acids have been exchanged.

15. The peptide of any one of claims 4 to 14, wherein the peptide comprises or consists of SEQ ID NO:2.

16. The agonist of any one of the preceding claims, wherein the peptide comprises or consists of SEQ ID NO:1.

17. The agonist of any one of the preceding claims, wherein the peptide comprises or consists of a sequence selected from the group consisting of SEQ ID NO:21. SEQ ID NO:22, SEQ ID NO:23, SEQ ID NO:24, SEQ ID NO:25, SEQ ID NO:26 and SEQ ID NO:27.

18. The agonist according to any one of claims 3 to 17, wherein said peptide contains a C-terminal amidation or an N-terminal acetylation.

19. The agonist of any one of the preceding claims, wherein the conjugated moiety is selected from a group consisting of peptides, lipids, glycans, saccharides and PEG.

20. The agonist of any one of the preceding claims, wherein the conjugated moiety is a saccharide, such as a mannose.

21. The agonist of any one of the preceding claims, wherein the conjugated moiety is a mannose, and wherein said mannose is conjugated to the N-terminal amino acid of the peptide.

22. The agonist of any one of the preceding claims, wherein the conjugated moiety is a fatty acid selected from the group consisting of Capric acid, Lauric acid, Myristic acid, Palmitic acid, Stearic acid, and Arachidic acid.

23. The agonist of any one of the preceding claims, wherein the conjugated moiety is a fatty acid linked to an amino acid, selected from the group consisting of -Naa-Capric acid, -Naa-Lauric acid, -Naa-Myristic acid, -Naa-Palmitic acid, -Naa-Stearic acid, and -Naa-Arachidic acid, wherein -Naa- is any proteinogenic amino acid.

24. The agonist of any one of the preceding claims, wherein the conjugated moiety is attached to a terminal amino acid or to a non-terminal amino acid of the peptide of any one of claims 5 to 18.

25. The agonist of any one of the preceding claims, wherein the conjugated moiety is selected from the group consisting of -Naa-Myristic acid, -Naa-Palmitic acid, -Naa-Stearic acid, wherein -Naa- is any proteinogenic amino acid, and wherein said conjugated moiety is attached to the Lysine in position 2 of a peptide of SEQ ID NO:1.

26. The agonist of any one of the preceding claims, wherein the conjugated moiety is selected from the group consisting of Myristic acid, Palmitic acid, Stearic acid, and wherein said conjugated moiety is attached to the N-terminal amino acid of a peptide of SEQ ID NO:1.

27. The agonist of any one of the preceding claims, wherein the conjugated moiety is a mannose, and wherein said mannose is conjugated to the N-terminal Serine of a peptide of SEQ ID NO:21.

28. The agonist of any one of the preceding claims, wherein the agonist has higher affinity to Tacr2 than to Tacr1.

29. The agonist of any one of the preceding claims, wherein the diabetes is any one of type 1 diabetes, type 2 diabetes and gestational diabetes.

30. The agonist of any one of the preceding claims, wherein said agonist reduces the blood glucose Cₘᵢₙ of said individual in need thereof. 30 31. The agonist of any one of the preceding claims, wherein said agonist increases the time interval between reaching the blood glucose Cₘᵢₙ in response to insulin and returning to pre-insulin glucose levels in said individual in need thereof.

32. A pharmaceutical composition comprising an agonist of Tacr2 or a pharmaceutically acceptable salt thereof as defined in any one of claims 1 to 31 for treatment of obesity, insulin resistance and/or diabetes in an individual in need thereof.

33. Use of an agonist of Tacr2 or a pharmaceutically acceptable salt thereof for manufacture of a medicament for treatment of insulin resistance, diabetes and/or obesity.

34. The use of claim 3333, wherein the agonist is as defined in any one of claims 1 to 31.

35. The use of claim 3333, wherein the diabetes is any one of type 1 diabetes, type 2 diabetes and gestational diabetes.

36. A method for treatment of insulin resistance, diabetes and/or obesity in an individual in need thereof, wherein the method comprises administering a therapeutically effective amount of an agonist of Tacr2 or a pharmaceutically acceptable salt thereof to said individual.

37. A method for treatment of insulin resistance, diabetes and/or obesity in an individual in need thereof, wherein the method comprises enhancing activity of brown adipose tissue cells in said individual by administration of a therapeutically effective amount of an agonist of Tacr2 or a pharmaceutically acceptable salt thereof to said individual.

38. A method for inducing weight loss in an individual in need thereof, wherein the method comprises administering a therapeutically effective amount of an agonist of Tacr2 or a pharmaceutically acceptable salt thereof to said individual.

39. A method for inducing weight loss in an individual in need thereof, wherein the method comprises enhancing activity of brown adipose tissue cells in said individual by administration of a therapeutically effective amount of an agonist of Tacr2 or a pharmaceutically acceptable salt thereof to said individual.

40. The method according to any one of claims 36 to 39, wherein the agonist of Tacr2 is as defined in any one of claims 2 to 42.

41. The method of any one of claims 36 to 40, wherein the agonist is administered parenterally to said individual.

42. The method of any one of claims 36 to 41, further comprising binding of said peptide agonist to said Tacr2.

43. The method of any one of claims 36 to 42, further comprising increasing basal oxygen consumption rate and/or norepinephrine-induced oxygen consumption rate and/or maximal oxygen consumption rate in primary brown adipocytes.

44. The method of any one of claims 36 to 43, further comprising increasing the absorption of glucose in brown and beige adipocytes.

45. The method of any one of claims 36 to 44, further comprising increasing the energy consumption in brown and beige adipocytes.

46. The method of any one of claims 36 to 45, further comprising increasing the insulin sensitivity in said individual.

47. The method of any one of claims 47 to 57, further comprising stimulating the release of endogenous neurokinin A in brown and beige adipocytes.

48. The method of any one of claims 36 to 47, further comprising reducing the blood glucose Cₘᵢₙ of said individual.

49. The method of any one of claims 36 to 48, further comprising increasing the time interval between reaching the blood glucose Cₘᵢₙ in response to insulin and returning to pre-insulin glucose levels.

50. The method of any one of claims 36 to 49, wherein diabetes is any one of type 1 diabetes, type 2 diabetes and gestational diabetes.
